(19) United States
(12) Patent Application Publication
(10) Pub. No.: US 2006/0275794 A1
(43) Pub. Date: Dec. 7, 2006

(54) Collections of matched biological reagents and methods for identifying matched reagents

(75) Inventors: John Carrino, San Diego, CA (US); Feng Liang, San Diego, CA (US)

Correspondence Address: BIOTECHNOLOGY LAW GROUP;C/O PORTFOLIOIP, PO BOX 52050, MINNEAPOLIS, MN, 55402, US

(73) Assignee: Invitrogen Corporation, Carlsbad, CA (US)

(21) Appl. No.: 11/371,354

(22) Filed: Mar. 7, 2006

Related U.S. Application Data
(60) Provisional application No. 60/673,045, filed on Apr. 19, 2005.
(60) Provisional application No. 60/665,199, filed on Mar. 25, 2005.
(60) Provisional application No. 60/665,200, filed on Mar. 25, 2005.
(60) Provisional application No. 60/659,493, filed on Mar. 7, 2005.
(60) Provisional application No. 60/659,492, filed on Mar. 7, 2005.

(30) Foreign Application Priority Data
Apr. 22, 2005 (WO) PCT/US05/13914

(51) Int. Cl. C12Q 1/68; C07H 21/04

(52) U.S. Cl. 435/6; 536/24.3

(57) ABSTRACT

Provided herein are collections of matched biological reagents selected from a larger collection of biological reagents, wherein the collection of matched biological reagents relate to a biological element. Also provided are methods for selling an isolated biomolecule or biological research reagent in a collection of matched biological reagents, and methods for selecting an isolated biomolecule or biological research reagent from a collection of biological reagents.

Priority is claimed to U.S. patent application Ser. No. 10/830,074, filed 23 Apr. 2004, and entitled “Online Procurement of Biologically Related Products/Services Using Interactive Context Searching of Biological Information”; U.S. Provisional Application No. 60/651,390, filed 8 Feb. 2005 by John Carrino and entitled “Collections of Matched Biological Reagents and Methods for Identifying Matched Reagents”; U.S. Provisional Application Ser. No. 60/659,492, filed 7 Mar. 2005 by John Carrino and Feng Liang and entitled “Collections of Matched Biological Reagents and Methods for Identifying Matched Reagents”; U.S. Provisional Application Ser. No. 60/659,493, filed 7 Mar. 2005 by John Carrino and Feng Liang and entitled “Collections of Matched Biological Reagents and Methods for Identifying Matched Reagents”; U.S. Provisional Application Ser. No. 60/665,200, filed 25 Mar. 2005 by John Carrino and Feng Liang and entitled “Collections of Matched Biological Reagents and Methods for Identifying Matched Reagents”; U.S. Provisional Application Ser. No. 60/665,199, filed 25 Mar. 2005 by John Carrino and Feng Liang and entitled “Collections of Matched Biological Reagents and Methods for Identifying Matched Reagents”; U.S. Provisional Application filed 19 Apr. 2005 by John Carrino and Feng Liang having docket number INV-1005-PV7 and entitled “Collections of Matched Biological Reagents and Methods for Identifying Matched Reagents”; U.S. Provisional Application No. 60/587,941, filed 14 Jul. 2004, and entitled “Methods and Systems for in Silico Experimental Design and for Providing-a Biotechnology Product to a Customer”; U.S. Provisional Application No. 60/608,293, filed 8 Sep. 2004, and entitled “Methods and Systems for in Silico Experimental Design and for Providing a Biotechnology Product to a Customer”; U.S. Provisional Application No. 60/588,158, filed 14 Jul. 2004, and entitled “Method for Providing Protein Microarrays”; U.S. Provisional Application No. 60/591,541, filed 26 Jul. 2004, and entitled “Method for Providing Protein Microarrays”; U.S. Provisional Application No. 60/592,239, filed 28 Jul. 2004, and entitled “Method for Providing Protein Microarrays”; and U.S. Provisional Application No. 60/953,586, filed 15 Feb. 2005, and entitled “Methods for Providing Protein Microarrays”; U.S. patent application Ser. No. 11/112,933, filed Apr. 22, 2005, by John Carrino, Feng Liang, Siamek Baharloo, and Barry I. Schweitzer, and entitled “Collections of Matched Biological Reagents and Methods for Identifying Matched Reagents”; International PCT application number PCT/US2005/013914, filed Apr. 22, 2005, by John Carrino, Feng Liang, Siamek Baharloo, and Barry I. Schweitzer, and entitled “Collections of Matched Biological Reagents and Methods for Identifying Matched Reagents”; which are all referred to and incorporated herein by reference in their entirety.

### FIELD OF THE INVENTION

The invention is in general directed to collections of biological reagents matched to one or more search criteria, and methods for identifying a sub-group of reagents matched to one or more search criteria from a larger collection.

### INCORPORATION BY REFERENCE

The present application also incorporates by reference herein, in its entirety, each of the following files encoded on a recordable compact disk (CD-R) filed herewith on 7 Mar. 2006: Table 1, which is contained in the file named “Table 1,” (size 3,427 KB, created Feb. 10, 2005), Table 2, which is contained in the file named “Table 2” (size 7,350 KB, created Feb. 10, 2005), Table 3, which is contained in the file named “Table 3” (size 4,037 KB, created Feb. 10, 2005), Table 4, which is contained in the file named “Table 4” (size 2 KB, created Feb. 10, 2005), Table 5, which is contained in the file named “Table 5” (size 63 KB, created Feb. 10, 2005), Table 6, which is contained in the file named “Table 6” (size 3 KB, created Feb. 10, 2005), Table 7, which is contained in the file named “Table 7” (size 70 KB, created Feb. 10, 2005), Table 8, which is contained in the file named “Table 8” (size 4 KB, created Feb. 10, 2005), Table 9, which is contained in the file named “Table 9” (size 849 KB, created Feb. 10, 2005), Table 10, which is contained in the file named “Table 10” (size 2680 KB, created Mar. 4, 2005), Table 11, which is contained in the file named “Table 11” (size 14987 KB, created Mar. 4, 2005), Table 12, which is contained in the file named “Table 12” (size 2051 KB; created Mar. 25, 2005), and Table 13, which is contained in the file named “Table 13” (size 1,316 KB; created Mar. 25, 2005). Each of these files is included on the CD-R filed herewith in duplicate labeled as “Copy 1” and “Copy 2.”

### SUMMARY

Provided herein is a collection of matched biological reagents comprising biomolecules and/or biological research products, comprising at least 100 different isolated biomolecules and/or biological research products of each of at least two biomolecule classes and/or biological research product classes. The matched biological reagents of the collection often are related to one or more biological elements (e.g., one or more search elements), such as a target biomolecule, a target biomolecular pathway, a target biomolecular pathway member, a disease, a disease pathway, and a disease pathway member. The biological reagents may, for example, be selected from the group consisting of antibodies, RNAi, nucleic acids, enzymes, proteins, cell culture products, detection products, separation media, microarrays, and the like. The collection sometimes comprises, for example, at least 10, at least 20, at least 25, at least 50, at least 100, at least 200, at least 250, at least 500, at least 750, at least 1000, at least 1100, at least 1200, at least 1300, at least 1400, at least 1500, at least 1750, at least 2000, at least 2250, at least 2500, at least 2750, at least 3000, at least 3500, at least 4000, at least 4500, at least 5000, at least 5500, at least 6000, at least 6500, at least 7000, at least 7500, at least 8000, at least 8500, at least 9000, at least 9500, or at least 10,000 different isolated biomolecules of each of at least 2, at least 3, at least 4, at least 5, at least 6, at least 7, at least 8, at least 9, or at least 10 biomolecule classes and/or biological research product classes. In some embodiments, the collection comprises at least 100 different isolated biomolecules of each of at least three biological research product classes. The collection sometimes comprises at least 50, at least 100, at least 150, at least 200, at least 250, at least 300, at least 350, at least 400, at least 450, at least 500, at least 750, or at least 1000 different isolated mammalian biomolecules. In certain embodiments, the collection comprises at least 100 different isolated nucleic acids, at least 100 different isolated proteins encoded by the at least 100 different isolated nucleic acids, at least 100 different antibodies against the at least 100 different proteins, and at least 100 different recombinant cell lines comprising each of the at least 100 different isolated nucleic acids. In certain embodiments, the collection comprises at least 50, at least 100, at least 150, at least 200, at least 250, at least 300, at least 350, at least 400, at least 450, at least 500, at least 750, or at least 1000 different isolated nucleic acids; at least 50, at least 100, at least 150, at least 200, at least 250, at least 300, at least 350, at least 400, at least 450, at least 500, at least 750, or at least 1000 different isolated proteins encoded by the at least 100 different isolated nucleic acids; at least 50, at least 100, at least 150, at least 200, at least 250, at least 300, at least 350, at least 400, at least 450, at least 500, at least 750, or at least 1000 different antibodies against the at least 100 different proteins; and at least 50, at least 100, at least 150, at least 200, at least 250, at least 300, at least 350, at least 400, at least 450, at least 500, at least 750, or at least 1000 different recombinant cell lines comprising each of the at least 50, at least 100, at least 150, at least 200, at least 250, at least 300, at least 350, at least 400, at least 450, at least 500, at least 750, or at least 1000 different isolated nucleic acids. In some embodiments, the collection comprises at least at least 1, at least 2, at least 3, at least 4, at least 5, at least 6, at least 7, at least 8, at least 9, at least 10, at least 15, at least 20, at least 25, at least 30, at least 40, at least 50, at least 75, at least 100, at least 150, at least 200, at least 250, at least 300, at least 350, at least 400, at least 450, at least 500, at least 750, or at least 1000 isolated proteins. In some embodiments, the collection comprises at least at least 1, at least 2, at least 3, at least 4, at least 5, at least 6, at least 7, at least 8, at least 9, at least 10, at least 15, at least 20, at least 25, at least 30, at least 40, at least 50, at least 75, at least 100, at least 150, at least 200, at least 250, at least 300, at least 350, at least 400, at least 450, at least 500, at least 750, or at least 1000 isolated proteins, such as, for example, the isolated proteins listed in the accompanying Table 13. In some embodiments, the collection comprises at least 1, at least 2, at least 3, at least 4, at least 5, at least 6, at least 7, at least 8, at least 9, at least 10, at least 15, at least 20, at least 25, at least 30, at least 40, at least 50, at least 75, at least 100, at least 150, at least 200, at least 250, at least 300, at least 350, at least 400, at least 450, at least 500, at least 750, or at least 1000 isolated proteins categorized as one family or class of proteins, for example, such as the families and classes listed in the accompanying Table 12. In some embodiments, the isolated proteins represent at least 5%, at least 10%, at least 15%, at least 20%, at least 25%, at least 30%, at least 40%, at least 50%, at least 60% at least 70%, at least 80%, or at least 90% of all members of a family or class of proteins, for example, such as the families and classes listed in the accompanying Table 12. A matched reagent collection may include, for example, matched reagents for each of at least 1, at least 2, at least 3, at least 4, at least 5, at least 6, at least 7, at least 8, at least 9, at least 10, at least 15, at least 20, at least 25, at least 30, at least 40, at least 50, at least 75, at least 100, at least 150, at least 200, at least 250, at least 300, at least 350, at least 400, at least 450, at least 500, at least 750, or at least 1000 isolated proteins. A matched reagent collection may include, for example, matched reagents for each of at least 1, at least 2, at least 3, at least 4, at least 5, at least 6, at least 7, at least 8, at least 9, at least 10, at least 15, at least 20, at least 25, at least 30, at least 40, at least 50, at least 75, at least 100, at least 150, at least 200, at least 250, at least 300, at least 350, at least 400, at least 450, at least 500, at least 750, or at least 1000 isolated proteins, such as, for example, those listed in Table 13. A matched reagent collection may include, for example, matched reagents for at least 1, at least 2, at least 3, at least 4, at least 5, at least 6, at least 7, at least 8, at least 9, at least 10, at least 15, at least 20, at least 25, at least 30, at least 40, at least 50, at least 75, at least 100, at least 150, at least 200, at least 250, at least 300, at least 350, at least 400, at least 450, at least 500, at least 750, or at least 1000 isolated proteins categorized as one family or class of proteins. A matched reagent collection may include, for example, matched reagents for isolated proteins that proteins represent at least 5%, at least 10%, at least 15%, at least 20%, at least 25%, at least 30%, at least 40%, at least 50%, at least 60% at least 70%, at least 80%, or at least 90% of all members of a family or class of proteins. Isolated proteins may be, for example, isolated native proteins, isolated recombinant native proteins, or isolated recombinant proteins with post-translational modifications.

Also provided is a method for selling an isolated biomolecule or biological research reagent, comprising: presenting to a customer an input function for identifying a target biological molecule or target biological pathway; and presenting to the customer a purchasing function comprising links to purchases of at least 10, at least 20, at least 25, at least 50, at least 100, at least 200, at least 250, at least 500, at least 750, at least 1000, at least 1100, at least 1200, at least 1300, at least 1400, at least 1500, at least 1750, at least 2000, at least 2250, at least 2500, at least 2750, at least 3000, at least 3500, at least 4000, at least 4500, at least 5000, at least 5500, at least 6000, at least 6500, at least 7000, at least 7500, at least 8000, at least 8500, at least 9000, at least 9500, or at least 10,000 different individual or different combinations of matched biological reagents of a collection of matched biological reagents comprising at least 10, at least 20, at least 25, at least 50, at least 100, at least 200, at least 250, at least 500, at least 750, at least 1000, at least 1100, at least 1200, at least 1300, at least 1400, at least 1500, at least 1750, at least 2000, at least 2250, at least 2500, at least 2750, at least 3000, at least 3500, at least 4000, at least 4500, at least 5000, at least 5500, at least 6000, at least 6500, at least 7000, at least 7500, at least 8000, at least 8500, at least 9000, at least 9500, or at least 10,000 different isolated biological reagents of each of at least 2, at least 3, at least 4, at least 5, at least 6, at least 7, at least 8, at least 9, or at least 10 biomolecule classes and/or biological research product classes, wherein the isolated biological reagents of the collection are related to the target biomolecule or biomolecular pathway. Certain embodiments are directed to a method for selecting an isolated biomolecule or biological research reagent, comprising: inputting a search parameter into an input function; identifying at least 10, at least 20,at least 25, at least 50, at least 100, at least 200, at least 250, at least 500, at least 750, at least 1000, at least 1100, at least 1200, at least 1300, at least 1400, at least 1500, at least 1750, at least 2000, at least 2250, at least 2500, at least 2750, at least 3000, at least 3500, at least 4000, at least 4500, at least 5000, at least 5500, at least 6000, at least 6500, at least 7000, at least 7500, at least 8000, at least 8500, at least 9000, at least 9500, or at least 10,000 different individual or different combinations of matched biological reagents from a collection of biological reagents comprising at least 10, at least 20, at least 25, at least 50, at least 100, at least 200, at least 250, at least 500, at least 750, at least 1000, at least 1100, at least 1200, at least 1300, at least 1400, at least 1500, at least 1750, at least 2000, at least 2250, at least 2500, at least 2750, at least 3000, at least 3500, at least 4000, at least 4500, at least 5000, at least 5500, at least 6000, at least 6500, at least 7000, at least 7500, at least 8000, at least 8500, at least 9000, at least 9500, or at least 10,000 different biological reagents of each of at least two biomolecule classes and/or biological research product classes, wherein the isolated biological reagents of the collection are related to the search parameter. The search parameter sometimes is selected from the group consisting of a target biological molecule, a target biological pathway, a target biological pathway member, a disease, a disease pathway, and a disease pathway member. The search parameter may also be based on gene ontology, wherein a target biological molecule is searched based on its protein or gene family or class. The biological reagents sometimes are selected from the group consisting of antibodies, RNAi, nucleic acids, enzymes, proteins, cell culture products,-detection products, separation media, microarrays, and the like. In some embodiments, the collection comprises at least 500 different isolated biological reagents of each of at least 2, at least 3, at least 4, at least 5, at least 6, at least 7, at least 8, at least 9, or at least 10 biomolecule classes and/or biological research product classes. The collection sometimes comprises at least 100 different isolated biological reagents of each of at least three biological research product classes, and sometimes the collection comprises at least 50, at least 100, at least 150, at least 200, at least 250, at least 300, at least 350, at least 400, at least 450, at least 500, at least 750, or at least 1000 different isolated mammalian biomolecules. In certain embodiments, the collection comprises at least 50, at least 100, at least 150, at least 200, at least 250, at least 300, at least 350, at least 400, at least 450, at least 500, at least 750, or at least 1000 different isolated nucleic acids; at least 50, at least 100, at least 150, at least 200, at least 250, at least 300, at least 350, at least 400, at least 450, at least 500, at least 750, or at least 1000 different isolated proteins encoded by the at least 50, at least 100, at least 150, at least 200, at least 250, at least 300, at least 350, at least 400, at least 450, at least 500, at least 750, or at least 1000 different isolated nucleic acids; at least 50, at least 100, at least 150, at least 200, at least 250, at least 300, at least 350, at least 400, at least 450, at least 500, at least 750, or at least 1000 different antibodies against the at least 50, at least 100, at least 150, at least 200, at least 250, at least 300, at least 350, at least 400, at least 450, at least 500, at least 750, or at least 1000 different isolated proteins; and at least 50, at least 100, at least 150, at least 200, at least 250, at least 300, at least 350, at least 400, at least 450, at least 500, at least 750, or at least 1000 different recombinant cell lines comprising each of the at least 50, at least 100, at least 150, at least 200, at least 250, at least at least 300, at least 350, at least 400, at least 450, at least 500, at least 750, or at least 1000 different isolated nucleic acids.

### BRIEF DESCRIPTION OF THE DRAWINGS

FIG. 1. Illustration of networked computer system.

FIG. 2. Illustration of data set entry.

FIG. 3. Window for Shopping Cart/Purchase Order.

FIG. 4. Window for search browser.

FIG. 5. Flow chart for processing search.

FIG. 6. Block diagram of Index File and File Map.

FIG. 7. Illustration of network search flow for Keyword, Sequence and ID searching.

FIG. 8. Flow chart for Purchase processing.

FIG. 9. Flow chart for processing keyword search.

FIG. 10. Browser window for Keyword and/or ID search.

FIG. 11. Results window for Keyword search.

FIG. 12. Results window for ID search.

FIG. 13. Browser window for Sequence search.

FIG. 14. Results window for Sequence search.

FIG. 15. Browser window for Ontology search.

FIG. 16. Illustration of network search flow for Gene Ontology searching.

FIG. 17. Table of examples of siRNA reagents that may be comprised in collections of matched biological reagents. Information for each siRNA in the table is organized in the following order: siRNA designation, catalog no., target gene symbol, definition, primary target accession, other target accession identifiers.

FIG. 18. List of examples of cell culture products that may be comprised in collections of matched biological reagents.

FIG. 19. A diagrammatical rendition of a non-limiting list of various types of matched reagents that can be included in a collection and methods of the present invention.

### DETAILED DESCRIPTION

Provided are collections of biological reagents matched to one or more input biological elements. Such collections of matched biomolecules and/or biological reagents are generated by sorting a larger collection of such molecules by one or more search parameters. These collections of matched reagents and methods for selecting them are useful in part for identifying a subset of research products from a larger collection of products that are suited to effecting a particular research objective. Such collections of matched reagents also are useful for selecting pertinent biological research reagents for purchase.

Certain terms utilized herein are defined hereafter.

Clone Collection: As used herein, “clone collection” refers to two or more nucleic acid molecules, each of which comprises one or more nucleic acid sequences of interest.

Customer: As used herein, the term customer refers to any individual, institution, corporation, university, or organization seeking to obtain genomic and proteomic products and services.

Provider: As used herein, the term provider refers to any individual, institution, corporation, university, or organization seeking to provide genomic and proteomic products and services.

Subscriber: As used herein, the term subscriber refers to any customer having an agreement with a provider to obtain public and private genomic and proteomic products and services at subscriber rates.

Non-subscriber: As used herein, the term non-subscriber refers to any customer who does not have an agreement with a provider to obtain public and private genomic and proteomic products and services at subscriber rates.

Host: As used herein, the term “host” refers to any prokaryotic or eukaryotic (e.g., mammalian, insect, yeast, plant, avian, animal, etc.) cell and/or organism that is a recipient of a replicable expression vector, cloning vector or any nucleic acid molecule. The nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Sambrook, et al., Molecular Cloning: A Laboratory Manual, Cold Spring Harbor Laboratory, Cold Spring Harbor, N.Y.

Related products or services. As used herein, the phrase “related product or service” refers to a product or service that relates to a region of a biomolecule, or an entire biomolecule, presented to a customer. A directly related product or service is a product or service that relates to an entire biomolecule presented to a customer. For example, if an in silico vector design experiment is design of a primer, then a link to a service for synthesizing the primer presented to the customer by the in silico primer design function, is a directly related product.

As used herein, the phrase “indirectly related product” refers to a product that relates to a region or feature of a biomolecule presented to a customer, but is not an entire biomolecule presented to a customer. In one embodiment of the invention, an indirectly related product refers to a portion or feature of the entire biomolecule, but the product is less then the entire biomolecule. In another embodiment, the indirectly related product may be peripheral to the specifically identified biomolecule, but related to the identified biomolecule in the sense that the product or service is useful and/or necessary in accomplishing the ultimate experimental goals of the researcher. For example, in an in silico vector design experiment, a link to an indirectly related product may be a link to the purchase of an antibiotic that corresponds to an antibiotic resistance gene that is on a vector that is designed by the in silico biotechnology experiment design and simulation function. A Table listing exemplary features and associated products is attached hereto (Table 2). From the specific product listing, general classes of products are revealed that can be used with the methods provided herein.

The phrase “indirectly related service” refers to a service that relates to a step, biomolecule, portion of a biomolecule, or feature of a biomolecule, provided by an in silico design or simulation experiment, but is not an entire step of the in silico design or simulation experiment that resulted in the presentation of the service to the customer. Furthermore, an indirectly related service can be related to a region of a biomolecule presented to a customer by the in silico design and simulation function, but is not synthesis of the entire biomolecule present to the customer.

Collections of Biological Research Reagents

The term “biological reagents” as used herein generally refers to biomolecules and biological research products utilized in biological research procedures. Biomolecules include but are not limited to proteins, peptides, antibodies, nucleic acids, nucleotides and variants of the foregoing, for example. Biological research products include but are not limited to cell culture products, detection products, separation media and microarrays, for example. Further examples of biological research products include but are not limited to an open reading frame, structural gene or transcriptional unit, enzymes, buffers, substrates, cofactors, indicator molecules, bioassay, vectors, antibodies, peptides, synthetic nucleic acids (e.g., DNA and RNA primers), proteins, cloning reagents, PCR reagents, cell culture products, and reagents needed for bioassays. Biological reagents are described in greater detail hereafter. A collection can include any of the biological reagents disclosed at and linked to http address www.invitrogen.com/content.cfm?pageid=9365 on the date this patent application is filed, which collection is hereby incorporated by reference in its entirety.

Proteins, Peptides and Variants Thereof

A protein sometimes is a native full-length protein, a portion of the protein, a polypeptide or a peptide. A portion of a protein includes but is not limited to an N-terminus, C-terminus, extracellular region, intracellular region, transmembrane region, subunit, active site (e.g., nucleotide binding region or a substrate binding region), a domain (e.g., an SH2 or SH3 domain). A protein sometimes comprises a post-translational modification (e.g., phosphorylation, glycosylation or ubiquination), for example. Proteins may be categorized in families or classes based on, for example, function, such as those presented in, for example, Table 12, or, for example, based on activity, such as those having enzyme classifications (for example, for illustrative purposes only, a protein kinase family may have various classes of protein kinases, such as, for example, tyrosine kinases and serine/threonine kinases, each class can itself be further subdivided into narrower classes of kinases). In certain embodiments, the protein is a signal transduction factor, cell proliferation factor, apoptosis factor, angiogenesis factor, or cell interaction factor. Examples of cell interaction factors include but are not limited to cadherins (e.g., cadherins E, N, BR, P, R, and M; desmocollins; desmogleins; and protocadherins); connexins; integrins; proteoglycans; immunoglobulins (e.g., ALCAM, NCAM-1 (CD56), CD44, intercellular adhesion molecules (e.g., ICAM-1 and ICAM-2), LFA-1, LFA-2, LFA-3, LECAM-1, VLA-4, ELAM and N-CAM); selectins (e.g., L-selectin (CD62L), E-selectin (CD62e), and P-selectin (CD62P)); agrin; CD34; and a cell surface protein that is cyclically internalized or internalized in response to ligand binding. Examples of signal transduction factors include but are not limited to protein kinases (e.g., mitogen activated protein (MAP) kinase and protein kinases that directly or indirectly phosphorylate it, Janus kinase (JAK1), cyclin dependent kinases, epidennal growth factor (EGF) receptor, platelet-derived growth factor (PDGF) receptor, fibroblast-derived growth factor receptor (FGF), insulin receptor and insulin-like growth factor (IGF) receptor); protein phosphatases (e.g., PTP1B, PP2A and PP2C); GDP/GTP binding proteins (e.g., Ras, Raf, ARF, Ran and Rho); GTPase activating proteins (GAFs); guanine nucleotide exchange factors (GEFs); proteases (e.g., caspase 3, 8 and 9), ubiquitin ligases (e.g., MDM2, an E3 ubiquitin ligase), acetylation and methylation proteins (e.g., p300/CBP, a histone acetyl transferase) and tumor suppressors (e.g., p53, which is activated by factors such as oxygen tension, oncogene signaling, DNA damage and metabolite depletion). The protein sometimes is a nucleic acid-associated protein (e.g., histone, transcription factor, activator, repressor, co-regulator, polymerase or origin recognition (ORC) protein), which directly binds to a nucleic acid or binds to another protein bound to a nucleic acid.

Protein and peptides sometimes include D-amino acids, L-amino acids, natural amino acids, unnatural or non-classical amino acids, and/or alpha amino acid homologs (e.g., beta²-, beta³- and/or gamma-amino acids). Examples of non-classical amino acids include but are not limited to omithine (hereinafter referred to as Z), diaminobutyric acid (hereinafter referred to as B), norleucine (hereinafter referred to as O), pyrylalanine, thienylalanine, naphthylalanine, phenylglycine, alpha and alpha-disubstituted amino acids, N-alkyl amino acids, lactic acid, halide derivatives of natural amino acids such as trifluorotyrosine, p-X-phenylalanine (where X is a halide such as F, Cl, Br, or I), allylglycine, 7-aminoheptanoic acid, methionine sulfone, norleucine, norvaline, p-nitrophenylalanine, hydroxyproline, thioproline, methyl derivatives of phenylalanine (Phe) such as 4-methyl-Phe, pentamethyl-Phe, Phe (4-amino), Tyr (methyl), Phe (4-isopropyl), Tic (1,2,3,4-tetrahydroisoquinoline-3-carboxyl acid), diaminopropionic acid, Phe (4-benzyl), 4-aminobutyric acid (gamma-Abu), 2-aminobutyric acid (alpha-Abu), 6-aminohexanoic acid (epsilon-Ahx), 2-aminoisobutyric acid (Aib), 3-aminopropionic acid, norvaline, hydroxyproline, sarcosine, citrulline, homocitrulline, cysteic acid, t-butylglycine, t-butylalanine, phenylglycine, cyclohexylalanine, fluoroamino acids, designer amino acids such as beta-methyl amino acids, and the like.

Variant amino acid sequences sometimes include suitable spacer groups inserted between any two amino acid residues of the sequence, such as alkyl groups (e.g., methyl, ethyl or propyl groups) or amino acid spacers (e.g., glycine or beta-alanine). Peptide moieties sometimes comprise or consist of peptoids. The term “peptoids” refers to variant amino acid structures where the alpha-carbon substituent group is linked to the backbone nitrogen atom rather than the alpha-carbon (e.g., Simon et al., PNAS (1992) 89(20), 9367-9371 and Horwell, Trends Biotechnol. (1995) 13(4), 132-134).

In certain aspects, the proteins, peptides, and variants thereof, are encoded by a portion of, or the entire nucleotide sequence of each nucleotide sequence of a collection of nucleotide sequences that include at least 2, 3, 4, 5, 6, 7, 8, 9, 10, 15, 20, 25, 30, 35, 40, 45, 50, 75, 100, 125, 150, 175, 200, 250, 500, 750, 1000, 1250, 1500, 1750, 2000, 2500, 3000 or all the nucleic acid sequences listed in Table 1 or Table 2, filed herewith on a separate CD. Table 1, filed herewith on CD in the file named “Table 1,” lists the coding sequences encoding 3469 human proteins that have been expressed and isolated at Invitrogen, Inc. using an insect cell system. Table 2, filed herewith, includes coding sequences encoding approximately 7600 human proteins, that include the 3469 coding sequences of Table 1 and coding sequences which are available as part of a commercial clone collection (Invitrogen, Inc., Carlsbad, Calif.), that are similar to those used to express the 3469 clones. In certain embodiments, the proteins of the present invention include isolated forms of at least some of the proteins of Table 9 or Table 13 or the proteins are encoded by all or a portion of the nucleotide sequences of Table 3, or by the nucleotide sequences whose accession numbers are listed in Table 4, Table 5, Table 6, Table 7, or Table 8.

Using a high throughput method, open reading frames encoding the 3469 recombinant human fusion proteins encoded by the nucleotide sequence of Table 1, were cloned, expressed, purified and arrayed. The human cDNAs were cloned into a Gateway entry vector, completely sequence-verified, expressed as GST and/or 6×His-fusions in a high-throughput baculovirus-based system, and purified using affinity chromatography. The proteins having accession numbers listed in Table 7, Table 8, Table 9 and Table 13 have been cloned and expressed and purified at the concentration indicated in the Tables using a high-throughput insect cell expression system, as well.

Antibodies

An antibody sometimes is a complete immunoglobulin or an antibody fragment. Antibodies sometimes are IgG, IgM, IgA, IgE, or an isotype thereof (e.g., IgG1, IgG2a, IgG2b or IgG3), sometimes are polyclonal or monoclonal, and sometimes are chimeric, humanized or bispecific versions of such antibodies. Antibody fragments include but are not limited to Fab, Fab′, F(ab)′2, Dab, Fv and single-chain Fv (ScFv) fragments. Bifunctional antibodies sometimes are constructed by engineering two different binding specificities into a single antibody chain and sometimes are constructed by joining two Fab′ regions together, where each Fab′ region is from a different antibody (e.g., U.S. Pat. No. 6,342,221). Antibody fragments often comprise engineered regions such as CDR-grafted or humanized fragments. Antibodies sometimes are derivitized with a functional molecule, such as a detectable label (e.g., dye, fluorophore, radioisotope, light scattering agent (e.g., silver, gold)) or binding agent (e.g., biotin, streptavidin), for example.

Nucleic Acids, Nucleotides and Variants Thereof

A nucleic acid may comprise or consist of DNA (e.g., genomic DNA (gDNA) and complementary DNA (cDNA)) or RNA (e.g., mRNA, tRNA, rRNA, and siRNA). A nucleic acid sometimes comprises or is a clone, vector (e.g., expression vector, shuttle vector, in vitro transcription/translation vector), open reading frame, an untranslated region, a tRNA, a suppressor tRNA, an rRNA, a primer, and an oligonucleotide. A vector sometimes is a plasmid or is linear, and sometimes includes one or more of a selectable marker, an origin of replication, a promoter (e.g., RNA polymerase or DNA polymerase), a PCR primer hybridization site, a topoisomerase linkage site, a recombinase interaction site, a cap, an enhancer and one or more stop codons (e.g., amber stop codon). A nucleotide or nucleoside may be provided in the collection, as well as analogs thereof. In embodiments where the nucleic acid is a synthetic oligonucleotide, the oligonucleotide can be about 8 to about 50 nucleotides in length, often about 8 to about 35 nucleotides in length, and sometimes from about 10 to about 25 nucleotides in length. Nucleic acids may include, for example, any of the nucleic acids disclosed at and linked to http address http://orf.invitrogen.com/cgi-bin/ORF_Browser on the date this patent application is filed, which collection is hereby incorporated by reference in its entirety.

Nucleic acids may comprise or consist of analog or derivative nucleic acids, such as polyamide nucleic acids (PNA) and others exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 5,614,622; 5,739,314; 5,955,599; 5,962,674; 6,117,992; WIPO publications WO 00/56746, WO 00/75372 and WO 01/14398, and related publications. Analog or derivative nucleic acids may also include stealth siRNA or other synthetic forms of siRNA. The term “siRNA reagent” comprises siRNA as well as modified forms of siRNA that have additional properties, such as causing a reduced level of induction of the PKR/interferon response pathway, avoidance of stress response to siRNA, higher specificity, or greater stability, compared to non-modified siRNA.

Nucleic acid molecules which can be used in the practice of the invention include interfering RNAs (RNAi) and those which generate RNAi. RNAi is double-stranded RNA (dsRNA) which mediates degradation of specific mRNAs, and can also be used to lower or eliminate gene expression.

RNAi may be produced in cells in vivo or synthesized ex vivo and then introduced into cells. When such molecules are synthesized in cells, they will often be generated by transcription of one or more nucleic acid molecules (e.g., DNA or RNA). A considerable number of expression systems are commercially available and include the BLOCK-IT™ Inducible H1 Lentiviral RNAi System available from Invitrogen Corp., Carlsbad, Calif. (cat. no. K4925-00).

While nucleic acid molecules with any number of different chemical modifications may be used in the practice of the invention, one example of a chemically modified nucleic acid molecule which may be used in the practice of the invention is-STEALTH™ RNAi (Invitrogen Corp., Carlsbad, Calif.).

A considerable number of chemically modified nucleic acid molecules, as well as chemical modifications themselves are described in U.S. Patent Publication No. 2004/0014956 (application Ser. No. 10/357,529) and U.S. patent application Ser. No. 11/049,636, filed Feb. 2, 2005), the entire disclosures of which are incorporated herein by reference.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of inhibiting or down regulating gene expression or viral replication, for example by mediating RNA interference “RNAi” or gene silencing in a sequence-specific manner; see for example Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). There is no particular limitation in the length of siRNA as long as it does not show toxicity. Non limiting examples of siNA molecules of the invention are presented in Table 10 on the CD-R filed herewith.

For example, the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic intercations, and/or stacking interactions. In certain embodiments, the siNA molecules of the invention comprise nucleotide sequence that is complementary to nucleotide sequence of a target gene. In another embodiment, the siNA molecule of the invention interacts with nucleotide sequence of a target gene in a manner that causes inhibition of expression of the target gene.

The double-stranded RNA portions of siRNAs in which two RNA strands pair up are not limited to the completely paired ones, and may contain nonpairing portions due to mismatch (the corresponding nucleotides are not complementary), bulge (lacking in the corresponding complementary nucleotide on one strand), and the like. Nonpairing portions can be contained to the extent that they do not interfere with siRNA formation. The “bulge” used herein preferably comprise 1 to 2 nonpairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, the double-stranded RNA region of siRNAs in which two RNA strands pair up may contain both bulge and mismatched, which sum up to, preferably 1 to 7, more preferably 1 to 5 in number. The terminal structure of siRNA may be either blunt or cohesive (overhanging) as long as siRNA enables to silence the target gene expression due to its RNAi effect.

As used herein, siNA molecules need not be limited to those molecules containing only RNA, but further encompasses chemically-modified nucleotides and non-nucleotides. In addition, as used herein, the term RNAi is meant to be equivalent to other terms used to describe sequence specific RNA interference, such as post transcriptional gene silencing, translational inhibition, or epigenetics. For example, siNA molecules of the invention can be used to epigenetically silence genes at both the post-transcriptional level or the pre-transcriptional level. In a non-limiting example, epigenetic regulation of gene expression by siNA molecules of the invention can result from siNA mediated modification of chromatin structure to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

In certain aspects, the nucleic acids include a portion of (e.g., at least 50, 100, 150, 200, 250, 300, 400, 500, 600, 700, 800, 900, 1000, 1500, 2000 nucleotides), or the entire nucleotide sequence of each nucleotide sequence of a collection of nucleotide sequences that include at least 2, 3, 4, 5, 6, 7, 8, 9, 10, 15, 20, 25, 30, 35, 40, 45, 50, 75, 100, 125, 150, 175, 200, 250, 500, 750, 1000, 1250, 1500, 1750, 2000, 2500, or 4000 or all the nucleic acid sequences listed in Table 1 or Table 2, filed herewith on a separate CD. Table 1, filed herewith on CD in the file named “Table 1,” lists the coding sequences encoding 3469 human proteins that have been expressed and isolated using an insect cell system. Table 2, filed herewith on CD in the file named “Table 2,” includes coding sequences encoding approximately 7600 human proteins, that include the 3469 coding sequences of Table 1, and coding sequences which are available as part of a commercial clone collection (Invitrogen, Inc., Carlsbad, Calif.).

In certain aspects, the nucleic acids encode at least some of the proteins of Table 9, Table 12, or Table 13, or the nucleotides include all or a portion of the nucleotide sequences of Table 3, or the nucleotide sequences whose accession numbers are listed in Table 4, Table 5, and Table 6. Alternatively, the nucleotide sequences of the nucleic acids of the present invention, encode all or a portion of the proteins whose accession numbers are listed in Table 7, Table 8, Table 9, Table 12, or Table 13.

Nucleic acid molecules of the invention include those which are engineered, for example, to produce dsRNAs. Examples of such nucleic acid molecules include those with a sequence that, when transcribed, folds back upon itself to generate a hairpin molecule containing a double-stranded portion. One strand of the double-stranded portion may correspond to all or a portion of the sense strand of the mRNA transcribed from the gene to be silenced while the other strand of the double-stranded portion may correspond to all or a portion of the antisense strand. Other methods of producing dsRNAs may be used, for example, nucleic acid molecules may be engineered to have a first sequence that, when transcribed, corresponds to all or a portion of the sense strand of the mRNA transcribed from the gene to be silenced and a second sequence that, when transcribed, corresponds to all or portion of an antisense strand (i.e., the reverse complement) of the mRNA transcribed from the gene to be silenced.

Nucleic acid molecules which mediate RNAi may also be produced ex vivo, for example, by oligonucleotide synthesis. Oligonucleotide synthesis may be used for example, to design dsRNA molecules, as well as other nucleic acid molecules (e.g., other nucleic acid molecules which mediate RNAi) with one or more chemical modification (e.g., chemical modifications not commonly found in nucleic acid molecules such as the inclusion of 2′-O-methyl, 2′-O-ethyl, 2′-O-propyl, 2′-fluoro, etc. groups).

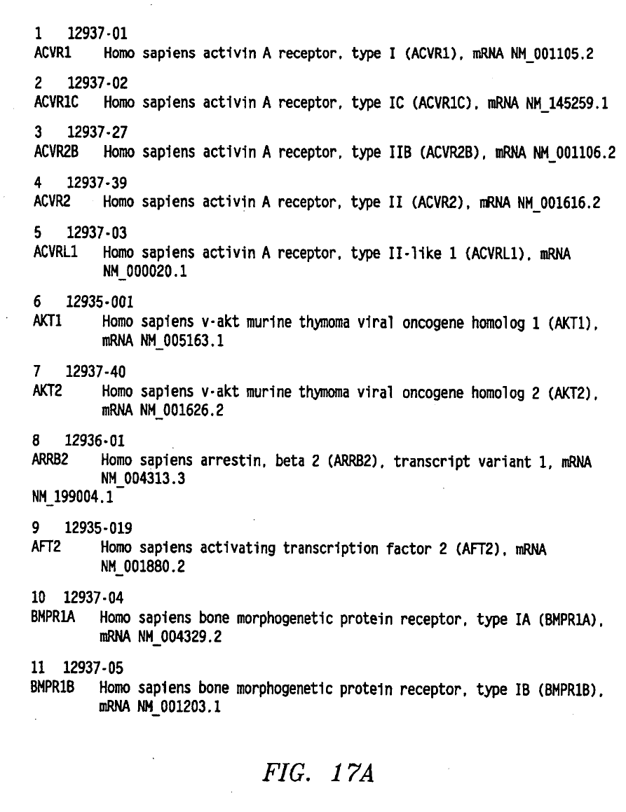

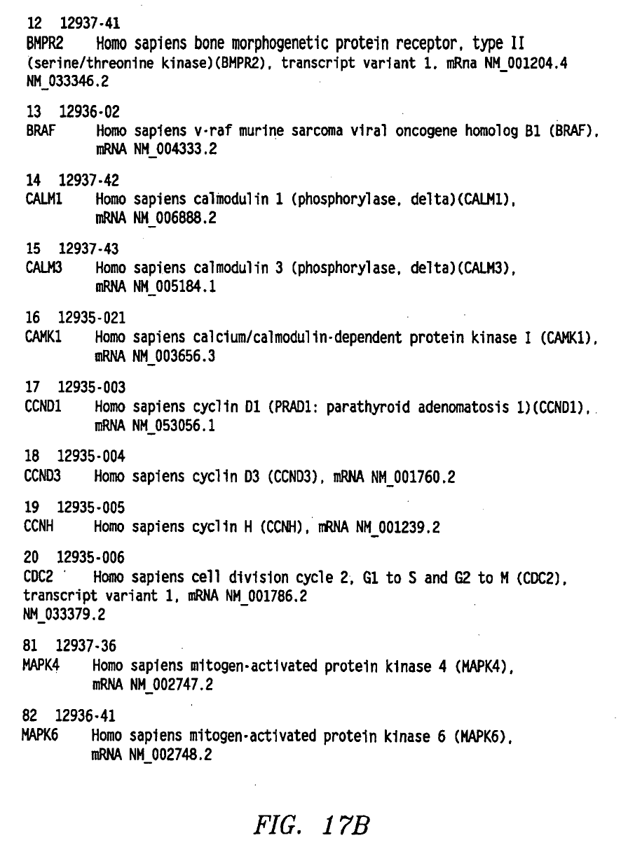

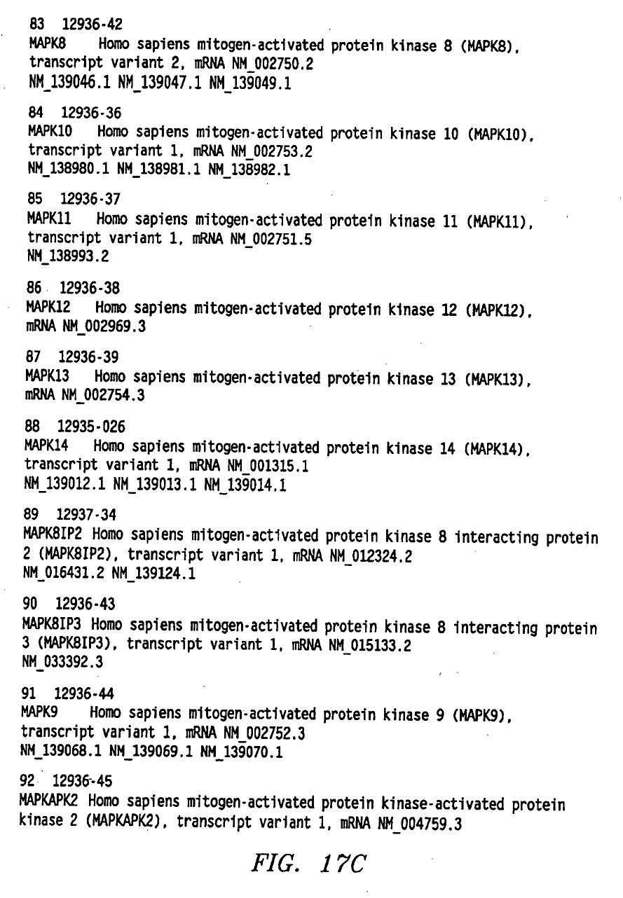

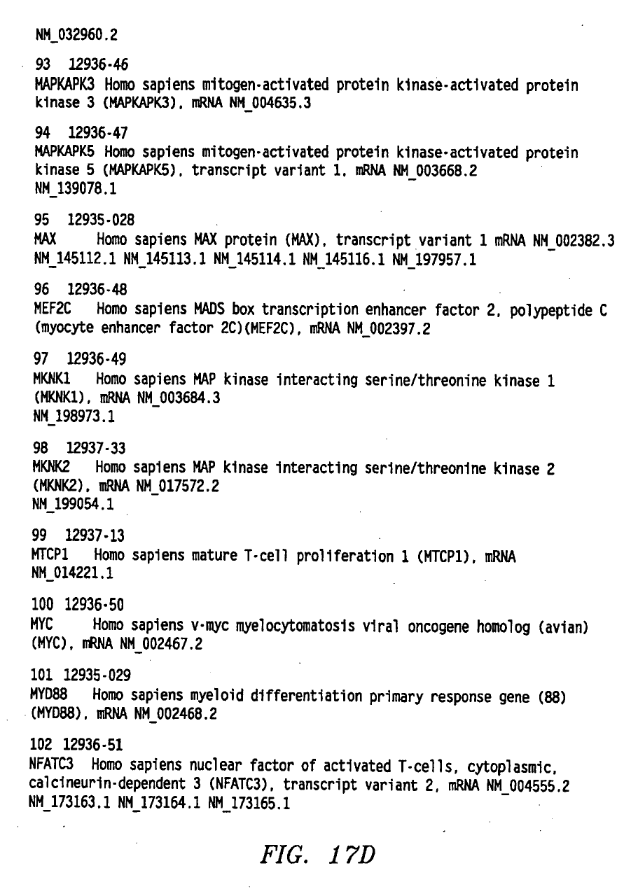

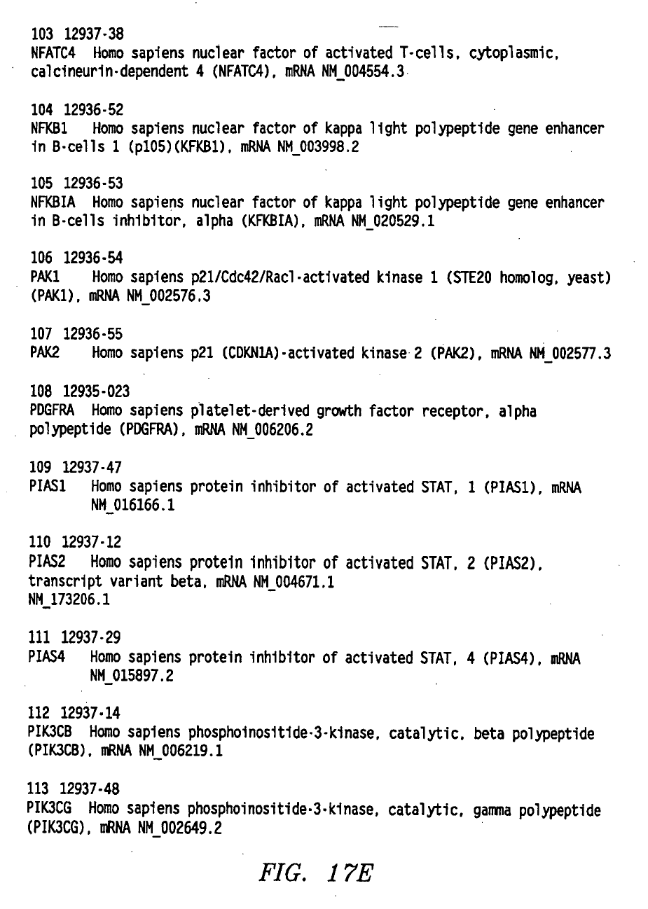

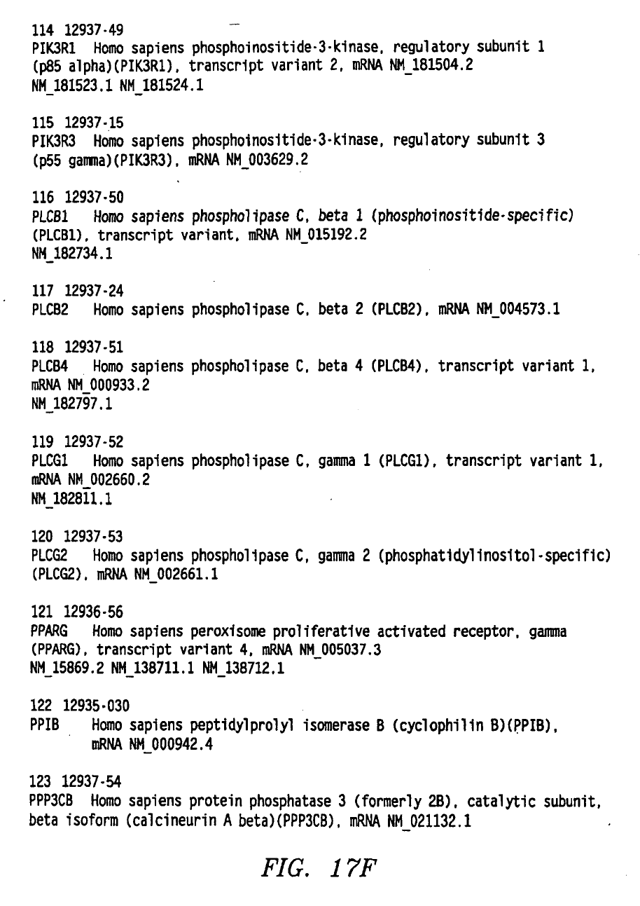

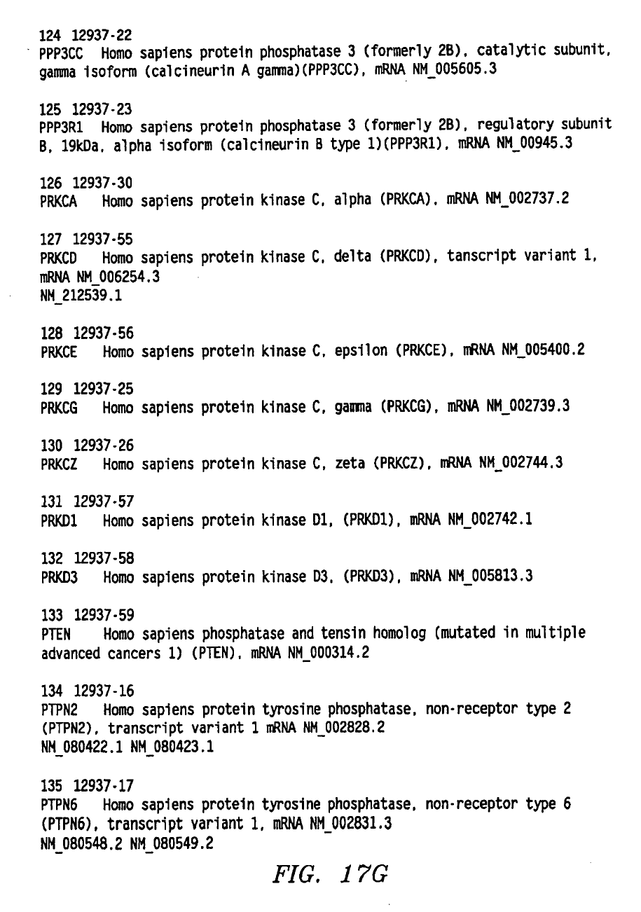

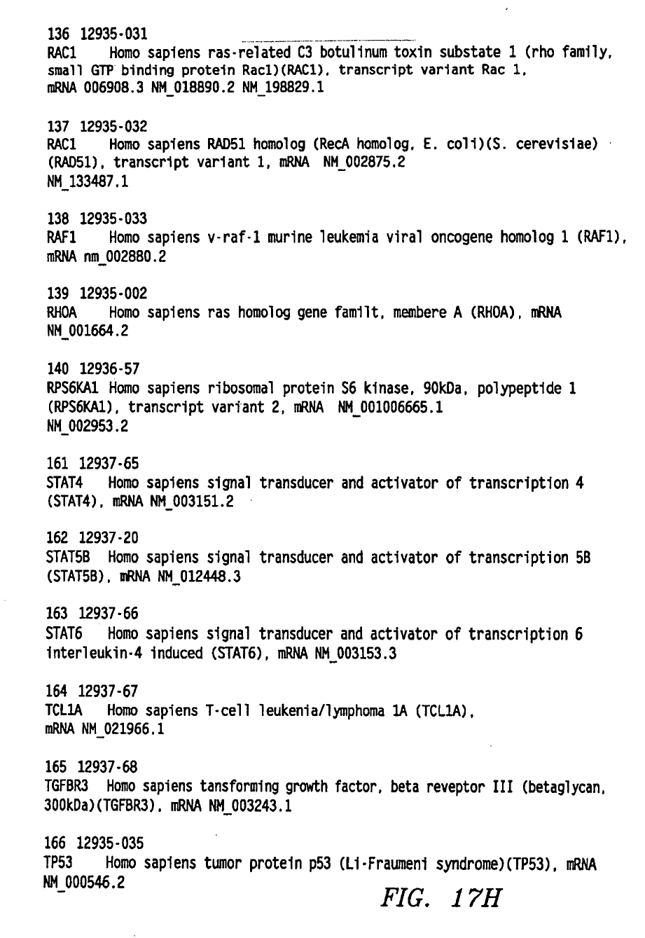

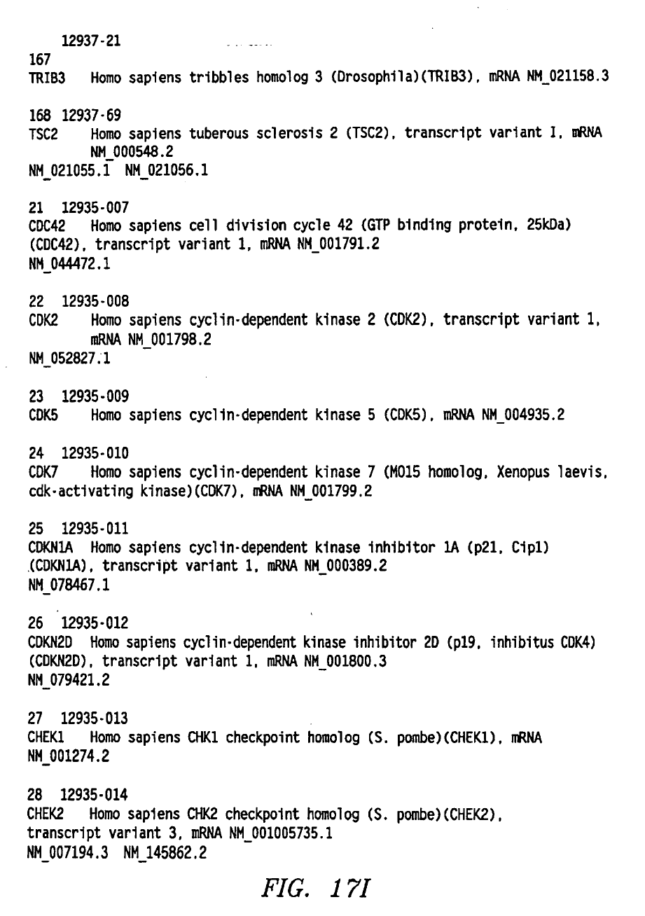

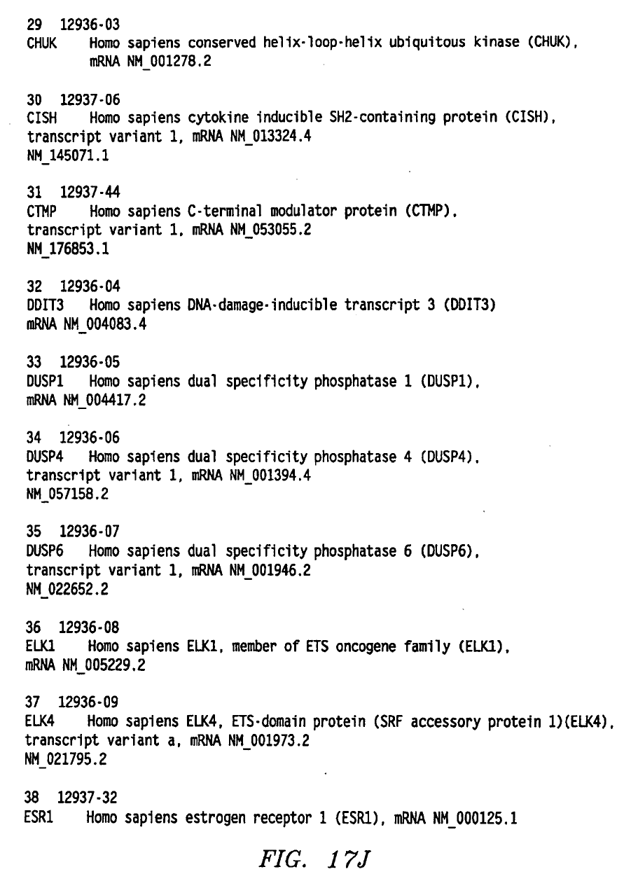

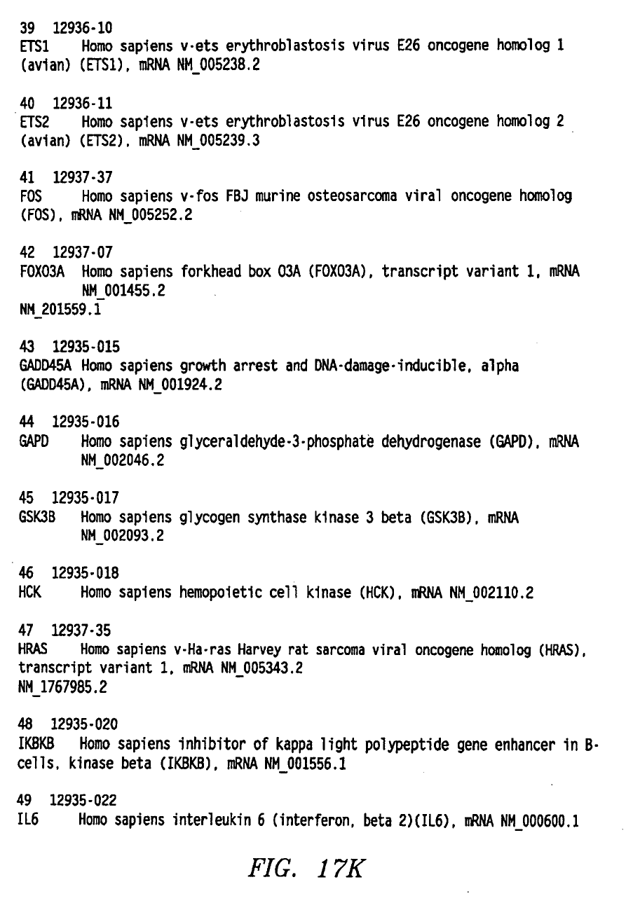

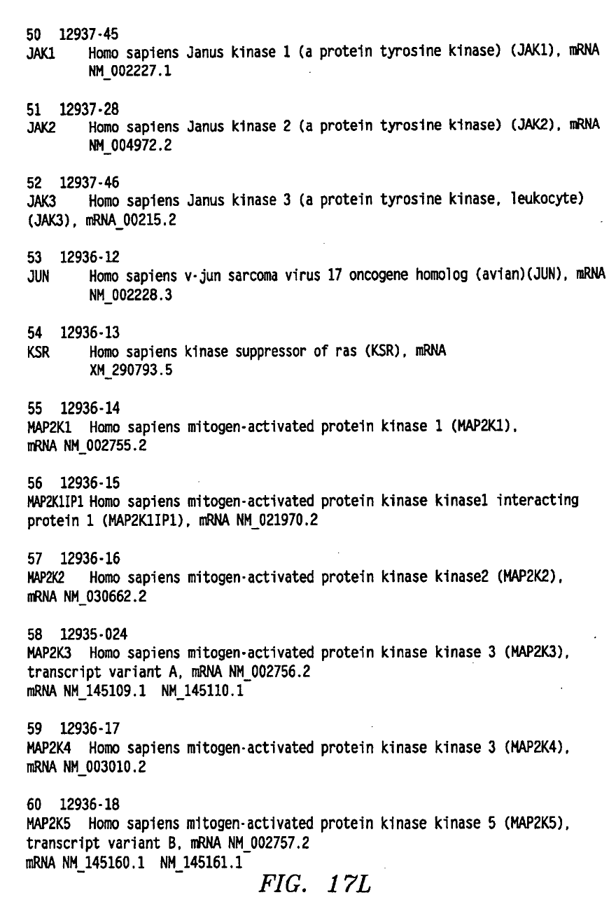

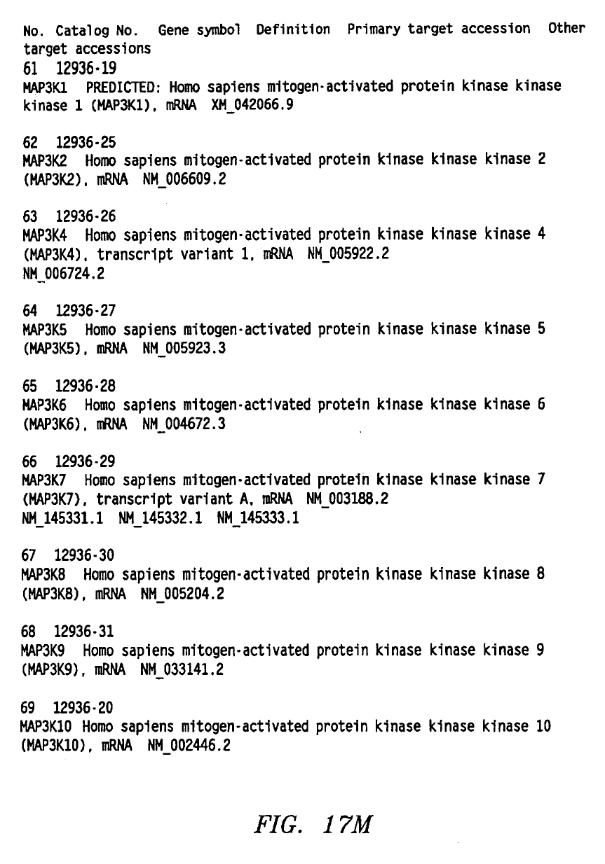

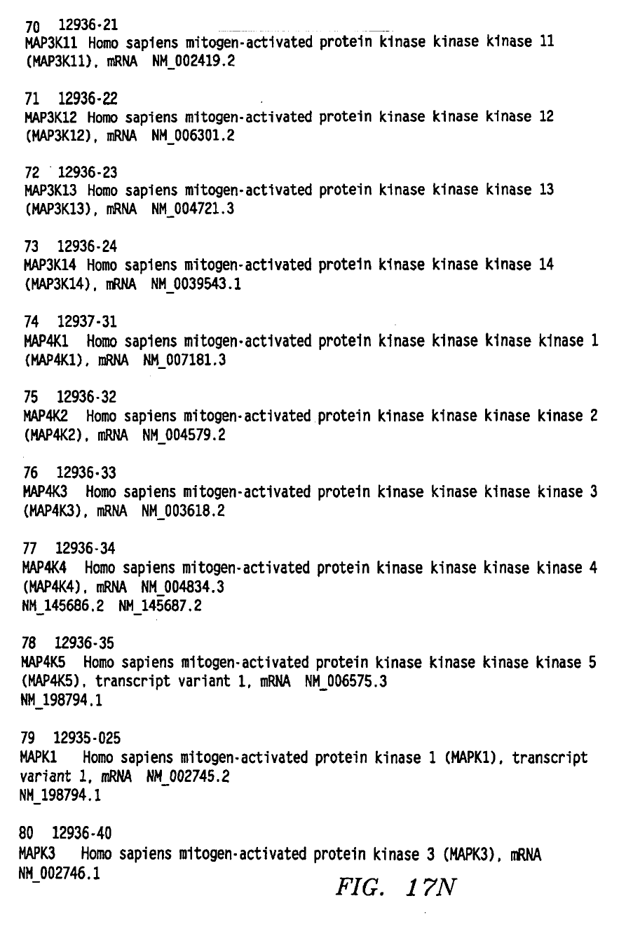

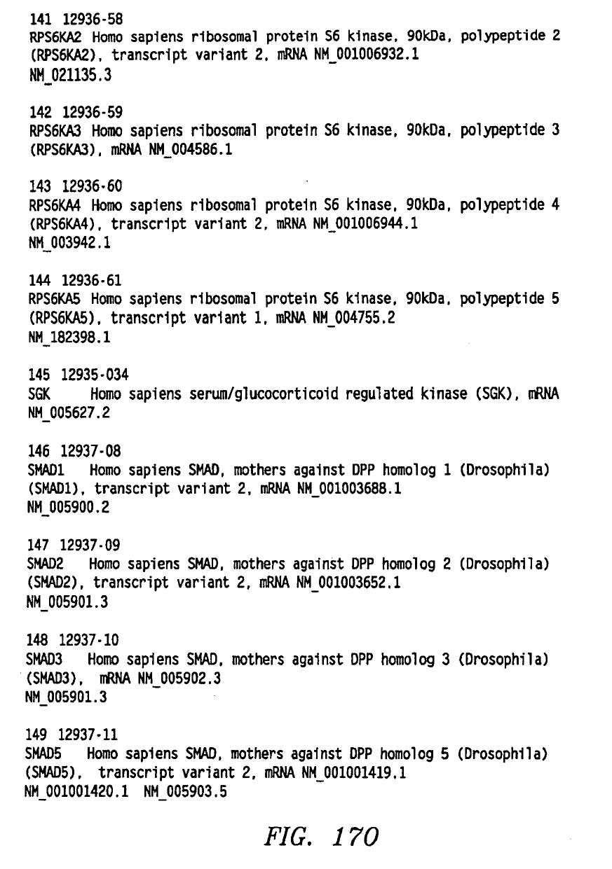

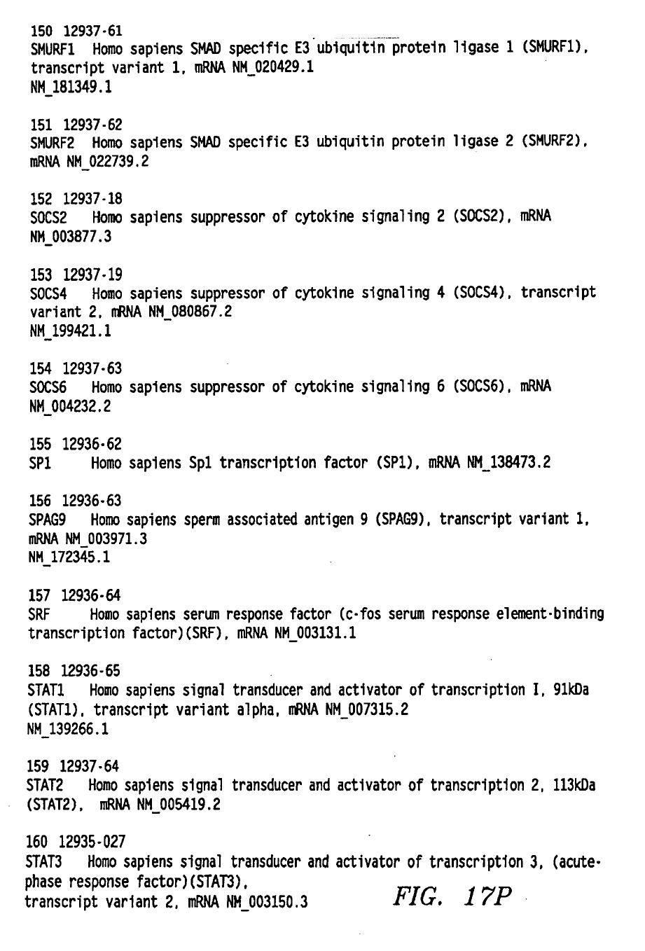

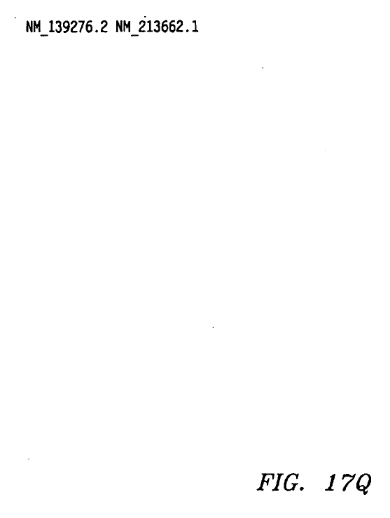

In some embodiments, a dsRNA to be used to silence a gene may have one or more (e.g., one, two, three, four, five, six, etc.) regions of sequence homology or identity to a gene to be silenced. Regions of homology or identity may be from about 20 bp (base pairs) to about 5 kbp (kilo base pairs) in length, 20 bp to about 4 kbp in length, 20 bp to about 3 kbp in length, 20 bp to about 2.5 kbp in length, from about 20 bp to about 2 kbp in length, 20 bp to about 1.5 kbp in length, from about 20 bp to about 1 kbp in length, 20 bp to about 750 bp in length, from about 20 bp to about 500 bp in length, 20 bp to about 400 bp in length, 20 bp to about 300 bp in length, 20 bp to about 250 bp in length, from about 20 bp to about 200 bp in length, from about 20 bp to about 150 bp in length, from about 20 bp to about 100 bp in length, from about 20 bp to about 90 bp in length, from about 20 bp to about 80 bp in length, from about 20 bp to about 70 bp in length, from about 20 bp to about 60 bp in length, from about 20 bp to about 50 bp in length, from about 20 bp to about 40 bp in length, from about 20 bp to about 30 bp in length, from about 20 bp to about 25 bp in length, from about 15 bp to about 25 bp in length, from about 17 bp to about 25 bp in length, from about 19 bp to about 25 bp in length, from about 19 bp to about 23 bp in length, or from about 19 bp to about 21 bp in length. Exemplary dsRNA molecules include those listed in Table 10 and also include those listed in FIG. 17. In some embodiments, dsRNA molecules are 40 base pairs or less, and contain 35 nucleotides, 30 nucleotides, 25 nucleotides, 20 nucleotides, 15 nucleotides, 10 nucleotides, or 5 nucleotides of a nucleotide sequence of Table 10 or FIG. 17. In some embodiments, dsRNA molecules are 35 base pairs or less in length, and contain 30 nucleotides, 25 nucleotides, 20 nucleotides, 15 nucleotides, 10 nucleotides, or 5 nucleotides of a nucleotide sequence of Table 10 or FIG. 17. In some embodiments, dsRNA molecules are 30 base pairs or less in length, and contain 25 nucleotides, 20 nucleotides, 15 nucleotides, 10 nucleotides, or 5 nucleotides of a nucleotide sequence of Table 10 or FIG. 17. In some embodiments, dsRNA molecules are 25 nucleotides or less in length, and contain 20 nucleotides, 15 nucleotides, 10 nucleotides, or 5 nucleotides of a nucleotide sequence of Table 10 or FIG. 17. In some embodiments, dsRNA molecules are 20 base pairs or less in length and contain 15 nucleotides, 10 nucleotides, or 5 nucleotides of a nucleotide sequence of Table 10 or FIG. 17. In some embodiments, dsRNA molecules are 15 base pairs or less in length and contain 10 nucleotides, or 5 nucleotides of a nucleotide sequence of Table 10 or FIG. 17. In some embodiments, dsRNA molecules are 15 base pairs or less in length and contain 10 nucleotides, or 5 nucleotides of a nucleotide sequence of Table 10 or FIG. 17. In some embodiments, dsRNA molecules are 10 base pairs or less in length and contain 5 nucleotides of a nucleotide sequence of Table 10 or FIG. 17. In some aspects, by containing a certain number of nucleotides is meant that the nucleotides are consecutive and have 100% identity. In certain aspects of the invention are provided isolated dsRNA biomolecules or at least one dsRNA biomolecule. DsRNA biomolecules include those, for example, listed in Table 10, FIG. 17, or any of the dsRNA biomolecules disclosed at and linked to http address rnaidesigner.invitrogen.com/dsRNA/searchValidatedStealth.jsp on the date this patent application is filed, which collection is hereby incorporated by reference in its entirety. In some aspects of the invention, are provided a collection comprising at least two, three, four, five, six, seven, eight, nine, ten, 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, or 700 different isolated biomolecules. In particular aspects of the invention are provided a collection of dsRNA biomolecules, comprising at least two, three, four, five, six, seven, eight, nine, ten, 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, or 700 different isolated dsRNA biomolecules. In some aspects, the dsRNA biomolecules are selected from the group consisting of the dsRNA biomolecules listed herein in Table 10 or FIG. 17. In some aspects, the dsRNA biomolecules are selected from the group consisting of the dsRNA biomolecules of the present application. In some aspects, the dsRNA biomolecules are selected from the group consisting of dsRNA biomolecules listed herein in Table 10 or FIG. 17, or any of the dsRNA biomolecules disclosed at and linked to http address rnaidesigner.invitrogen.com/dsRNA/searchValidatedStealth.jsp on the date this patent application is filed, which collection is hereby incorporated by reference in its entirety. In other aspects of the invention are provided pharmaceutical compositions comprising at least one dsRNA-biomolecule. In other aspects of the invention are provided collections of pharmaceutical compositions comprising two, three, four, five, six, seven, eight, nine, ten, 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, or 700 different pharmaceutical compositions, each pharmaceutical composition comprising at least one dsRNA biomolecule. In some aspects of the invention each pharmaceutical composition comprises at least one dsRNA biomolecule, wherein the dsRNA biomolecule is selected from the group consisting of dsRNA biomolecules listed herein in Table 10, FIG. 17, or any of the dsRNA biomolecules disclosed at and linked to http address rnaidesigner.invitrogen.com/dsRNA/searchValidatedStealth.jsp on the date this patent application is filed, which collection is hereby incorporated by reference in its entirety. In some aspects of the invention each pharmaceutical composition comprises at least one dsRNA biomolecule, wherein the dsRNA biomolecule is selected from the group consisting of dsRNA biomolecules listed in the present application. In some aspects, each pharmaceutical composition comprises at least one dsRNA biomolecule, wherein the dsRNA biomolecule is selected from the group consisting of dsRNA biomolecules listed herein in Table 10 or FIG. 17.

A hairpin containing molecule having a double-stranded region may be used as RNAi. The length of the double stranded region may be from about 20 bp (base pairs) to about 2.5 kbp (kilo base pairs) in length, from about 20 bp to about 2 kbp in length, 20 bp to about 1.5 kbp in length, from about 20 bp to about 1 kbp in length, 20 bp to about 750 bp in length, from about 20 bp to about 500 bp in length, 20 bp to about 400 bp in length, 20 bp to about 300 bp in length, 20 bp to about 250 bp in length, from about 20 bp to about 200 bp in length, from about 20 bp to about 150 bp in length, from about 20 bp to about 100 bp in length, 20 bp to about 90 bp in length, 20 bp to about 80 bp in length, 20 bp to about 70 bp in length, 20 bp to about 60 bp in length, 20 bp to about 50 bp in length, 20 bp to about 40 bp in length, 20 bp to about 30 bp in length, or from about 20 bp to about 25 bp in length. The non-base-paired portion of the hairpin (i.e., loop) can be of any length that permits the two regions of homology that make up the double-stranded portion of the hairpin to fold back upon one another.

Any suitable promoter may be used to control the production of RNA from the nucleic acid molecules of the invention. Promoters may be those recognized by any polymerase enzyme. For example, promoters may be promoters for RNA polymerase II or RNA polymerase III (e.g., a U6 promoter, an H1 promoter, etc.). Other suitable promoters include, but are not limited to, T7 promoter, cytomegalovirus (CMV) promoter, mouse mammary tumor virus (MMTV) promoter, metalothionine, RSV (Rous sarcoma virus) long terminal repeat, SV40 promoter, human growth hormone (hGH) promoter. Other suitable promoters are known to those skilled in the art and are within the scope of the present invention.

Double-stranded RNAs used in the practice of the invention may vary greatly in size. Further the size of the dsRNAs used will often depend on the cell type contacted with the dsRNA. As an example, animal cells such as those of C. elegans and Drosophila melanogaster do not generally undergo apoptosis when contacted with dsRNAs greater than about 30 nucleotides in length (i.e., 30 nucleotides of double stranded region) while mammalian cells typically do undergo apoptosis when exposed to such dsRNAs. Thus, the design of the particular experiment will often determine the size of dsRNAs employed.

In many instances, the double stranded region of dsRNAs contained within or encoded by nucleic acid molecules used in the practice of the invention will be within the following ranges: from about 20 to about 30 nucleotides, from about 20 to about 40 nucleotides, from about 20 to about 50 nucleotides, from about 20 to about 100 nucleotides, from about 22 to about 30 nucleotides, from about 22 to about 40 nucleotides, from about 20 to about 28 nucleotides, from about 22 to about 28 nucleotides, from about 25 to about 30 nucleotides, from about 25 to about 28 nucleotides, from about 30 to about 100 nucleotides, from about 30 to about 200 nucleotides, from about 30 to about 1,000 nucleotides, from about 30 to about 2,000 nucleotides, from about 50 to about 100 nucleotides, from about 50 to about 1,000 nucleotides, or from about 50 to about 2,000 nucleotides. The ranges above refer to the number of nucleotides present in double stranded regions. Thus, these ranges do not reflect the total length of the dsRNAs themselves. As an example, a blunt ended dsRNA formed from a single transcript of 50 nucleotides in total length with a 6 nucleotide loop, will have a double stranded region of 23 nucleotides.

As suggested above, dsRNAs used in the practice of the invention may be blunt ended, may have one blunt end, or may have overhangs on both ends. Further, when one or more overhang is present, the overhang(s) may be on the 3′ and/or 5′ strands at one or both ends. Additionally, these overhangs may independently be of any length (e.g., one, two, three, four, five, etc. nucleotides). As an example, STEALTH™ RNAi is blunt at both ends.

The invention also includes sets of RNAi and those which generate RNAi. Such sets include those which either (1) are designed to produce or (2) contain more than one dsRNA which directed against the same target gene. As an example, the invention includes sets of STEALTH™ RNAi wherein more than one STEALTH™ RNAi shares sequence homology or identity to different regions of the same target gene. The nucleotide sequences of exemplary STEALTH™ RNAi which may be used in the practice of the invention are set out in Table 10. Table 11 provides the nucleotide and encoded protein sequences that correspond to the RNAi sequences of Table 10.

Cell Culture Products

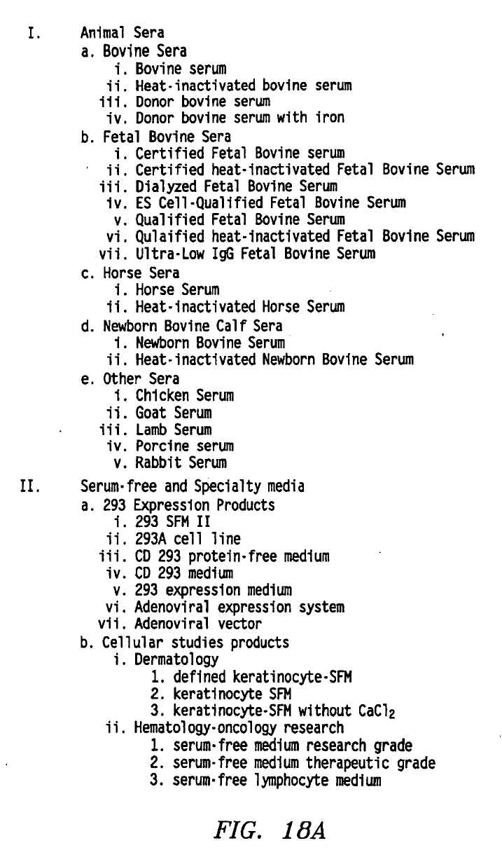

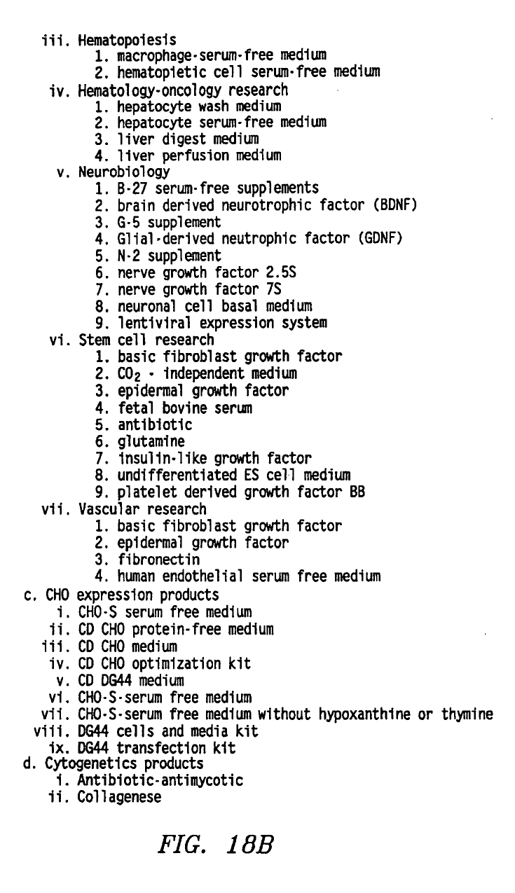

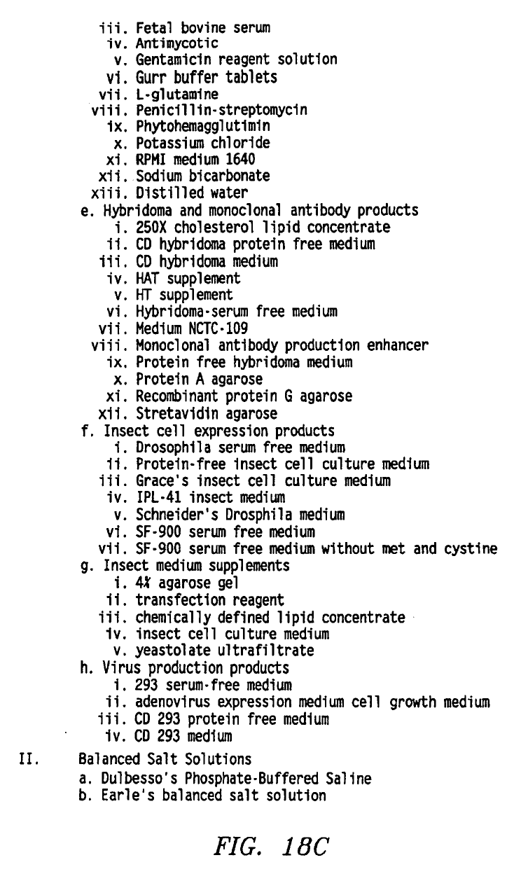

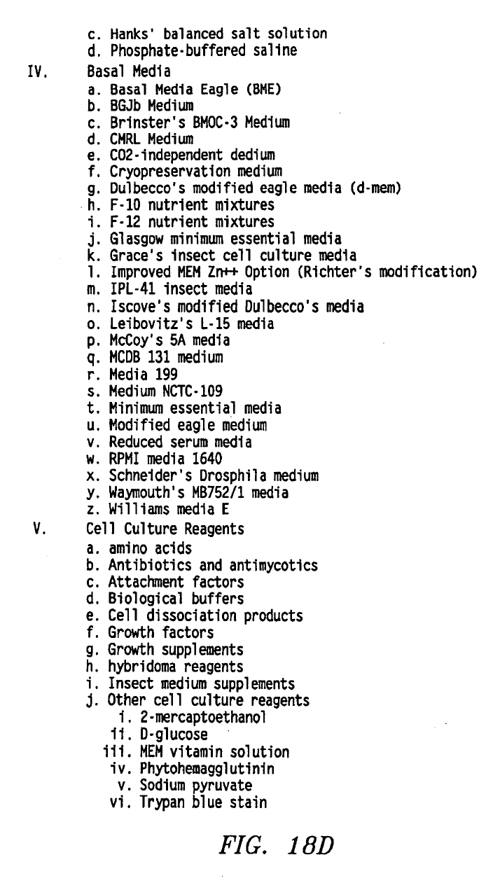

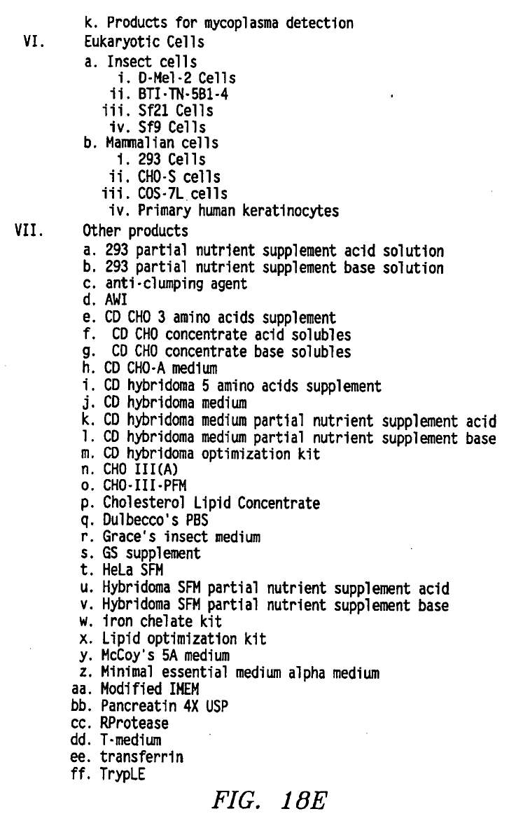

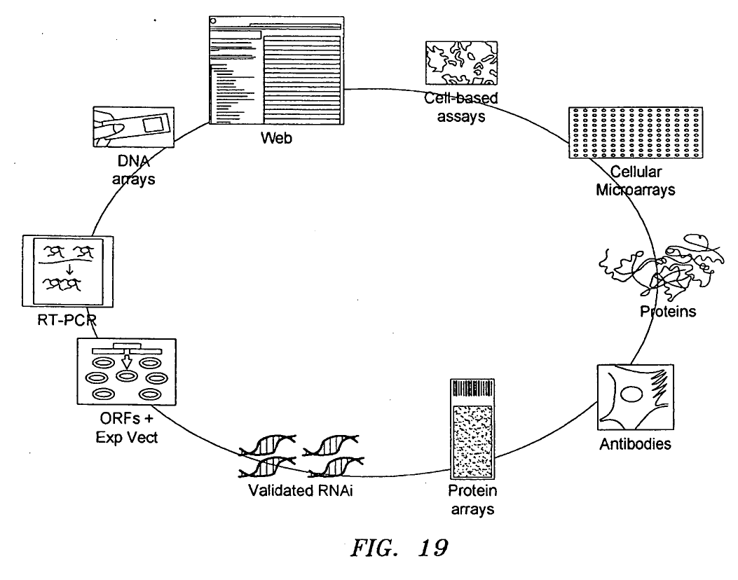

Cell culture products, including cells, cell-culture media, and cell culture components such as, for example, serum, nutrients, salts, antibiotics, and other additives, for growing and/or maintaining cells are provided (e.g., bacteria, yeast, insect and mammalian cells) in the collection. Culture media may be nutrient rich or nutrient poor, and sometimes is selected based on the cells grown or maintained. Also provided in the collection are cells (e.g., bacteria, yeast, insect and mammalian cells), including cells competent for transfection of a nucleic acid, and cells modified for use in cellular assays. Included in the collection are reagents and apparatus for transfecting a nucleic acid into a cell, such as detergents and electroporation devices, for example. Cell culture products also comprise vessels and apparatus for growing and/or maintaining cells, such as flasks, dishes, plates, and fermentors. Cell culture products may include, for example, those listed in FIG. 18.

Detection Products, Separation Media and Microarrays

Provided in the collection are detection products, including, for example, bioassays, and products used to perform bioassays, such as antibodies, including, for example, epitope-specific antibodies, detectable labels (e.g., fluorophores, radioisotopes, light scattering compounds (e.g., molecules containing gold or silver), dyes), metabolic labels, enzymatic labels, light-producing labels such as, for example, luciferase, and molecules capable of linking detection agents to a molecule (e.g., derivitized biotin or streptavidin). Bioassays may include, for example, in vitro assays and cell based assays. Other examples of detection products include ion indicators, such as calcium, magnesium, sodium, potassium, chloride, or heavy metal indicators, chelating agents, and pH indicators. Other examples of detection products include instrumentation used in bioassays and other assays, such as, for example, flow cytometers, mass spectrometers, and consumable and non-consumable products used with the instrumentation, such as, for example, tubes, flasks, slides, plates, microspheres, and nanospheres. Other examples of detection products include electrophoresis products such as gel electrophoresis instrumentation, supplies, pre-cast gels, blotting, such as Western blot products, standards, stains, and dyes. Detection products may include, for example, products to detect protein-protein interactions. A collection may include a planar solid support (microtiter plate with wells, wafer with pits or wells), a chromatography resin, a bead (e.g., magnetic bead) for separation of biomolecules. Such separation media sometimes are derivitized with affinity agents, such as ligands, analytes, proteins, and oligonucleotides. A collection often comprises one or more microarrays, sometimes high density microarrays, with arrays of nucleic acids and/or arrays of proteins or peptides. Microarrays also include, for example, cellular microarrays. Detection products may also include products such as those disclosed at and linked to the http address https://catalog.invitrogen.com/index.cfm?fuseaction=viewCatalog.viewCategories&npc=92&pc=2 32&nc=232 on the date this patent application is filed, which collection is hereby incorporated by reference in its entirety.

Databases, Search Elements, Search Interfaces and Database Output

As used herein, “procuring,” including grammatical variations thereof, means to obtain, gain, access, receive, acquire, or buy.

As used herein, “appropriate,” including grammatical variations thereof, means capable of being acted on or carrying out an act. For example, an appropriate request or command when inputted into a dialog box would trigger a search of a database to find or identify an object conforming to the request or command (e.g., keyword search to retrieve objects containing the inputted keyword).

As used herein, “biologically related,” including grammatical variations thereof, means associated with life and living processes. For example, anaerobic respiration is a biologically related metabolic action. Protein expression (in vitro) is another example.

As used herein, “electronic storage medium,” including grammatical variations thereof, means space in electronic memory where information is held for later use. For example, this may include, but is not limited to, magnetic tape, CD-ROMS, DVD, optical disks, flash drives, RAM or floppy disk.

As used herein, “electronic inventory,” including grammatical variations thereof, means a digital catalog which corresponds to some or all of the products and or services offered by the vendor.

As used herein, “target item,” including grammatical variations thereof, means data or files to be affected by an action. For example, a target item can be a file name, a word, an image, a text string, a number or a value stored on electronic media that is retrievable upon request by a user.

As used herein, “sundry groupings,” including grammatical variations thereof, means a collection of various data segregated into named files for orderly access of such data from an electronic storage medium.

As used herein, “interfacing,” including grammatical variations thereof, means the method of interaction between a person and a computer, or between a computer and a peripheral device, or between two computers. In a related aspect, user interface would include the environment that permits one to interact with a computer (e.g., World Wide Web, WiFi, browsers, web pages).

As used herein, “user,” including grammatical variations thereof, means an entity that requests services from a server. The entity can be a human or a device (e.g., see input devices, above).

As used herein, “user terminals,” including grammatical variations thereof, means a node or hardware that accesses a server.

As used herein, “bi-directional communication,” including grammatical variations thereof, means a process by which information is exchanged between two systems in both directions, where each system receives and sends information.

As used herein, “searchable,” including grammatical variations thereof, means the ability of data or files to be looked into in an effort to mark, find or discover such data or files.

As used herein, “extracts,” including grammatical variations thereof, means a product prepared by retrieving files or data from a database or server.

As used herein, “associated biological attributes,” including grammatical variations thereof, means a specific feature related to living things and/or processes of living things (including such a feature carried out in vitro).

As used herein, “request,” including grammatical variations thereof, means one or a series of user inputs or commands for retrieving information from a server or database.

As used herein, “inputting,” including grammatical variations thereof, means the act of entering a request or data. For example, typing at a keyboard pointing, speaking to, etc.

As used herein, “hierarchal menu output,” including grammatical variations thereof, means a list transmitted to the user (e.g., but not limited to, a display on a computer screen) of available alternatives for selection by the operator or user organized into orders or ranks each subordinate to the one above it.

As used herein, “display,” including grammatical variations thereof, means what a user sees on a CRT unit or monitor. More broadly, substitutes may be used as displays, such as auditory signals for the visually impaired or any other means of information communication.

As used herein, “subset,” including grammatical variations thereof, means a set each of whose elements is an element of an inclusive set.

As used herein, “empirical measure of similarity” including grammatical variations thereof, means a method of comparing target items or objects between extracts containing such items or objects, where the extracts are considered to be similar if the distance between the items or objects comprising the extracts is small according to arbitrary values of attributes or annotations associated with items or objects in the target file. For example, values can be given for molecular weights, isoelectric points, metabolic pathway participation, restriction maps, organisms, protease fragments, epitopes, hydropathic profiles, separation patterns, such as electrophoresis gels, chromatographic output, mass spec output, fluorescence data, tissue distributions, expression patterns, kinetic constants, binding constants, antagonists, agonists, inverse agonists, linkage maps, substrates, ligands, inhibitors, disease associations, alleles, homologies, interacting molecules, biological functions, phosphorylation patterns, sub-cellular localizations,-glycosylation patterns, post-translational modification patterns, motif consensus, crystal structures, pharmacokinetic properties, pharmacologic properties, and toxicologic properties secondary, tertiary and/or quaternary structures. Thus, for example, each attribute can be given a numerical value. Further, each biologically related product, for example, would have a different set of values for some or all of these attributes/annotations. Extracts with values for one or more attributes/annotations that are numerically similar are judged to be similar. Using such similarity, as distances between values become greater, the extracts are judged as less similar. Based on software design choices, ranks for the spectrum of similarity are determined and the resulting output of the extracts of interest are reflected in hierarchical fashion according to high and low values of similarity. Systems for determining such similarity are disclosed in, for example, U.S. Pat. No. 5,835,087, herein incorporated by reference.

As used herein, “graphic user interface (GUI),” including grammatical variations thereof, means a user interface to a computer that uses icons to represent items, such as documents and programs, that the user can access and manipulate with a pointing device or other signal transducer.

As used herein, “annotated text strings,” including grammatical variations thereof, means text or embedded comments or instructions within text which may or may not print but which may be viewed and referred to by an operator or user that include a consecutive series of characters to be specified by command.

As used herein, “base text,” including grammatical variations thereof, means the number of different values that can be represented by each digit position (e.g., binary or base 2) that correspond to the body copy on a page.

As used herein, “loci,” including grammatical variations thereof, means a site or one or more digital addresses where related information may be found.

As used herein, “objects,” including grammatical variations thereof, means a searchable element that is a part of a locus. For example, an annotation under an “organism” locus would be considered an object.

As used herein, “hyperlinks,” including grammatical variations thereof, means a pointer within a hypertext document that points (links) to another document, which may or may not be a hypertext document.

As used herein, “server,” including grammatical variations thereof, means a functional unit that provides shared services to workstations/clients/users over a network; for example, a file server, a print server, a mail server. The server may be internal or external, single or multitask.

As used herein, “Web page browser,” including grammatical variations thereof, means a program used to read a file or to navigate through a hypermedia document.

As used herein, “parsable,” including grammatical variations thereof, means to be amenable to analysis where the operands entered with a command create a parameter list in the command processor from the information.

As used herein, “sub-window,” including grammatical variations thereof, means a secondary window that is presented to a user to allow the user to perform a task on the primary browser window. For example, a dialog box is a sub-window.

As used herein, “module,” including grammatical variations thereof, means, a self-contained functional unit which is used with a larger system. For example, a software module is a part of a gram that performs a particular task.

As used herein, “word-for-word searching” including grammatical variations thereof, means a keyword or keywords serve as the primary unit that represents the information for which the search is being conducted, where the search systems will search for strings of words, as well as individual words. Such a system will not automatically keep words together as a phrase. Further, a word-for-word searching method would envisage the use of wild cards (i.e., include variant endings to any word request).

As used herein, “Boolean searching,” including grammatical variations thereof, means a search structure that uses the logical operators, AND, OR & NOT, to connect search terms in search statements. The operators tell the database what the relationship is between the search terms. Further, a Boolean searching method would envisage the use of wild cards (i.e., include variant endings to any word request).

As used herein, “proximity searching,” including grammatical variations thereof, means a search structure that uses relative location and distance of query words or characters in a search statement. The location and distance operators (e.g., “near,” “adjacent,” “within”) tell the database what the relationship is between the search terms. Further, a proximity searching method would envisage the use of wild cards (i.e., include variant endings to any word request).

As used herein, “phrase searching,” including grammatical variations thereof, means keywords serve as the primary unit that represents the information for which the search is being conducted, where the search systems will search for strings of words. Such a system will automatically keep words together as a phrase. Further, a phrase searching method would envisage the use of wild cards (i.e., include variant endings to any word request).

As used herein, “truncation,” including grammatical variations thereof, means a searching system that uses a symbol at the end of a word to retrieve variant endings of that word.

As used herein, “keyword jump,” including grammatical variations thereof, means a method of navigation that transports a user to content/record stored on a database by entering a keyword or code associated with that content/record.

As used herein, “Blast server,” including grammatical variations thereof, means Basic Local Alignment Search Tool, which is a set of similarity search programs designed to explore all of the available sequence databases regardless of whether the query is protein or nucleic acid.

As used herein, “gene ontology,” including grammatical variations thereof, means a controlled and dynamic vocabulary that can be applied to all organisms as knowledge of gene and protein roles in cells accumulates and changes.

As used herein, “public consortium,” including grammatical variations thereof, means an individual or group recognized by a community to possess authority that can be cited freely by members of the public and understood by members of the community.

As used herein, “tabbed,” including grammatical variations thereof, means a way of creating the DHTML dialog boxes, or the like (HTML, XHTML, XML), or sub-windows as a type of interfacing to load such sub-windows.

As used herein, “triggers,” including grammatical variations thereof, means to initiate, acuate, or set off a program.

As used herein, “tree navigation,” including grammatical variations thereof, means using an organization of directories (or folders) and files which resemble the branches of an upside-down tree that allow users to find their way through a Web site.

In some embodiments, provided herein are methods for selling an isolated biomolecule or biological research reagent or service, related thereto, that include: presenting to a customer an input function for identifying a target biological molecule or target biological pathway; and presenting to the customer a purchasing function comprising links to purchases of at least 100, 200, 250, 500, 750, 1000, 1250, 1500, 1750, 2000, 2500, 5000, 7500, 10000, 12500, 15000, 17500, 20000 different individual or different combinations of matched biomolecules and/or biological research products of a collection of matched biomolecules and/or biological research products comprising at least 100 different isolated biomolecules and/or biological research products of each of at least two biomolecule classes and/or biological research product classes, wherein the isolated biomolecules and/or biological research products of the collection are related to the target biomolecule or biomolecular pathway.

Methods of such embodiments are performed by a provider to generate revenue from a customer. Exemplary products offered by the provider can include clone collections and individual clones, polypeptides, such as enzymes, antibodies, libraries (e.g., cDNA libraries, genomic libraries, etc.), buffers, growth media, purification systems, primers, cell lines, chemical compounds, fluorescent labels, functional assays, and a variety of kits including DNA and protein purification, amplification and modification. Further, these exemplary products are provided for example only and are not intended to limit the present invention.

In certain aspects, provided is a method for selling a plurality of related products and services that generates revenue for a provider. Exemplary services offered by the provider include clone construction services, protein expression services, antibody production services, library (e.g., cDNA library, genomic library, etc.) construction services, and research and development consulting services.

One or more input functions described herein typically search a collection of biological reagents typically is searched by a biological element to identify a sub-collection of biological reagents matched to the biological element. A biological element includes but is not limited to a biomolecule, pathway or condition, and often is utilized to search a collection of biological reagents to identify matched reagents. The term “matched” as used herein generally refers to a collection of reagents related to the biological element. One or more biological elements may be utilized to search a collection, and can be selected from the group consisting of a target biomolecule, a target biomolecular pathway, a target biomolecular pathway member, a disease, a disease pathway, and a disease pathway member. A target biomolecule sometimes is a nucleic acid or protein, such as one or more of those described herein. The search parameter may also be based on gene ontology, wherein a target biological molecule is searched based on its protein or gene family. For example, a target protein that is a member of a protein class or family may be searched within that class or family. Protein classes or families may include, for example, G-protein coupled receptors, kinases, protein kinases, nuclear hormone receptors, protein phosphatases, phosphodiesterases, proteases such as, for example, endopeptidases and exopeptidases, ino channels, cytokines, and chemokines. Other examples of protein classes or families are listed in, for example, Table 12, which is incorporated by reference herein. Examples of expressed isolated proteins are listed in, for example, Table 13, which is incorporated by reference herein. A target biomolecular pathway often is a related group of biomolecules that interact with one another (e.g., bind, phosphorylate, dephosphorylate, cleave by proteolysis) in cells or tissues of an organism. A disease is any known condition or disorder, and a disease pathway often is a group of biomolecules that interact with one another in diseased tissue or cells. Additional examples of biological elements include but are not limited to biological attributes such as nucleic acid or amino acid sequences, molecular weights, isoelectric points, metabolic and signal pathway participation, restriction maps, organisms, protease fragments, epitopes, hydropathic profiles, separation patterns, such as electrophoresis gels, chromatographic output, mass spec output, fluorescence data, tissue distributions, expression patterns, kinetic constants, binding constants, antagonists, agonists, inverse agonists, linkage maps, substrates, ligands, inhibitors, disease associations, alleles, homologies, interacting molecules, biological functions, phosphorylation patterns, sub-cellular localizations, glycosylation patterns, post-translational modification patterns, motif consensus, crystal structures, pharmacokinetic properties, pharmacologic properties, toxicologic properties, secondary, tertiary and/or quaternary structures. In addition to one or more biological elements, customer information-can-be added to a purchase server customer database when there is not a match between the stored information and that contained in a customer name field.

Descriptors corresponding to a collection of physical biological reagents generally are maintained in one or more databases. Known database structures can be utilized for maintaining descriptors corresponding to the collection. Descriptors include but are not limited to a scientific name descriptive of a biological reagent, a commercial name descriptive of a biological reagent, a chemical representation of a biological reagent, an amino acid or nucleotide sequence corresponding to a biological reagent, a research protocol useful for using a biological reagent, a flow chart showing mechanisms of action for one or more reagents, and price information, for example. Further examples of descriptors include but are not limited to organisms, nucleotide accession numbers, related accession numbers, gene names, gene definitions, gene symbols, text summary of gene products, expression profiles, mRNA records, references, length of inserts in base pairs, nucleic acid sequences, collection names, collection types, vector names, vector antibiotics, host names, Stealth RNA, siRNA, protein accession numbers, protein records, amino acid sequences, molecular weights, isoelectric points, protease digestion patterns, domain searches, predicted secondary and tertiary structures, binding sites, classes of enzymes, classes of substrates, associated proteins (for example, other members of protein complexes), inhibitors, blockers, agonists, antagonists, labels, tags, markers or other indicators, protein model searches, Online Mendelian Inheritance in Man (OMIM) data, product data, metabolic pathway data, single nucleotide polymorphism (SNP) data, SNP map data, locus link ID, Unigene ID and genomic alignment data. Descriptors corresponding to a biological reagent often are linked to one or more descriptors corresponding to an input biological element utilized to search the collection, described in greater detail below.

A collection of biological research reagents generally is searched according to the methods provided herein, by inputting one or more biological elements into an input interface or input function. Input interfaces and input functions are known and are provided in a convenient apparatus, orientation and location for a user, and can be provided via a wide area network, such as an Internet portal. A biological element may be input by a variety of means, including but not limited to, manual input devices or direct data entry devices (DDEs). For example, manual devices may include, keyboards, concept keyboards, touch sensitive screens, light pens, mouse, tracker balls, joysticks, graphic tablets, scanners, digital cameras, video digitizers and voice recognition devices. DDEs may include, for example, bar code readers, magnetic strip codes, smart cards, magnetic ink character recognition, optical character recognition, optical mark recognition, and turnaround documents.

A user in a remote location often inputs information in the input interface, where access is remotely provided to an electronic data server-to a user-where the server receives input from a user and processes the input to produce a first output, based on interfacing with one or more public consortium databases, where the latter database has one or more databases which are, for example, proprietary to an offeror of the product or service. The user can select one or multiple products or services or ahyperlinkor description of a product or service to create an extract, where the extract serves as an output for the user, thus, facilitating delivery of a product or service to the user, whether delivery is remote or local to the offerer/user. In a related aspect, the choice of delivery may be that of the offerer or user. A server utilized sometimes is an in-house server, public server or other private server. For example, the public server may include a government institution, a private institution, a college or university, a consortium or a private individual. Other databases may include data related to inventory, shippers, seasonal or regional requirements, credit history, hazardous products and interactions, notifications associated with making dangerous or hazardous products, warning flags, and the like.

Any search or sorting feature useful for identifying a sub-group of biological reagents matched to a biological element from a larger collection can be utilized. Such searches include but are not limited to word-for-word searches, Boolean searches, proximity searches, phrase searches, truncation searches or a combination of the foregoing. In other embodiments, methods may include processing string searches using a BLAST server (including, but not limited to, in-house or external server) or keyword jump navigation. Further, such searches may include accessing external databases/servers. Search algorithms can include but are not limited to Dijkstra and Bellman-Ford algorithms, sometimes with skeletal or heuristic elements, for example.

A matched collection that is the output from a search is in a form useful to a user (e.g., a list, a table), and may include a set of descriptors selected by the user (e.g., price information and nucleotide sequences of research reagents). An output format also may include a relevance indicator that shows a user the degree of relation between an input parameter and a matched reagent in the output collection.

The resulting outputs may, for example, be displayed as browser pages containing for example, hierarchical menus that are based on the retrieved extracts which provide the user with one or more subsets or compilations of the stored target items. The menus represent assortments of target items within the subsets, where the content and/or format of the displayed target items is based on an empirical measure of similarity of the associated biological attributes for all of the assorted target items. Moreover, the hierarchical menu output display pages identify favored or all target items assorted into each of the files which have one or more associated biological attributes in common to enable a user, for example, to differentiate products and/or services of interest stored on electronic media and to obtain or purchase one or more listed products or services (i.e., custom order, catalog listing or service provided) by activating an appropriate graphic user interface (e.g., a check box) that is included on the displayed output pages. In one aspect, any one menu item output on the displayed format page will contain a buy option graphic user interface (GUI) and one or more of the following, including a clone identification number, definition of the expressed product, gene symbol, and accession number. For example, a hierarchical menu may, on the first page, provide the user with names of more than one target biomolecule from a matched biological reagent collection associated with a given pathway. Each of the target biomolecules may be linked to the next level of pages, with items from the matched biological reagent collection matched to each of the target biomolecules. In one example, when the user clicks the hyperlink, a list of products and/or services can be presented to the customer, or a series of options can be presented such as “isolated proteins,” “antibodies,” “nucleic acid probes,” “clones,” “biological research products,” “cell culture products,” or “services” that when selected link to Internet pages with the matched product and/or service that can be customized based on the identified target protein(s).

Or, after a search based on a disease, the output may first be a browser page listing multiple pathways or target biomolecules associated with that disease, with further links to additional matched biological reagents.

When the user clicks the hyperlink, a list of products and/or services can be presented to the customer, or a series of options can be presented such as “isolated proteins,” “antibodies,” “nucleic acid probes,” “clones,” or “services” that when selected link to Internet pages with the matched product and/or service that can be customized based on the identified target protein(s).

Convenient and useful database structures, input interfaces, search algorithms, output formats, user interfaces, and information transmission systems are known and described elsewhere, such as in U.S. patent application Ser. No. 10/830,074, filed 23 Apr. 2004 by Feng Liang, entitled “Online procurement of biologically related products/services using interactive context searching of biological information,” incorporated in its entirety by reference. U.S. patent application No. 60/591,541, filed 26 Jul. 2004 by Paul Predki et al., entitled “Methods for providing protein microarrays,” incorporated in its entirety by reference; and U.S. patent application No. 60/608,293, filed 8 Sep. 2004 by Siamak Baharloo et al., entitled “Methods and systems for in silico experimental design and for providing a biotechnology product to a customer,” Incorporated in its entirety by reference.

The purchasing function included in methods provided herein can provide one or more hyperlinks to related products or services. The purchasing function allows the customer to purchase the related products or services presented to the customer after the customer identifies a target biological molecule or target biological pathway. The purchasing function can be linked to an Internet based shopping cart. Therefore, the customer upon being presented with links for purchasing related biotechnology products, can click the links to learn more about the biotechnology products and/or to add the related biotechnology products to an Internet shopping cart. Therefore, the provider generates revenue when the purchaser purchases the one or more products and/or services using the purchasing function

Examples of database structures, input interfaces, search algorithms, output formats, user interfaces, and information transmission systems, are presented in the Examples.

### EXAMPLES

The examples set forth below illustrate but do not limit the invention.

### Example 1

### Collections of Biological Reagents Comprising siRNA Reagents

Collections of biological reagents may comprise, for example, siRNA reagents. Those of ordinary skill in the art will recognize that the present example relating to siRNA reagents may be used to exemplify collections of biological reagents that comprise, for example, other nucleic acids, proteins, and antibodies. Collections of biological reagents may comprise, for example, siRNA and siRNA reagents presented herein in Table 10 or in FIG. 17, or any of the siRNA reagents disclosed at and linked to http address rnaidesigner.invitrogen.com/sima/searchValidatedStealth.jsp on the date this patent application is filed, which collection is hereby incorporated by reference in its entirety. A collection of biological reagents comprising siRNA reagents may be searched, for example, by inputting a search term into an input interface or input function. Such search terms may include, for example, any term that may be used to identify the siRNA, its target, the cellular pathway comprising the target, or diseases associated with the target or the cellular pathway. Search terms may include, for example, a gene symbol, accession number, key word, locus ID, Unigene ID, catalog number, target biological molecule, target biological pathway, disease, disease pathway, disease pathway member, cellular process, or a nucleotide sequence. Examples of input search terms include NCBI gene accession numbers, such as those having formats NM_{—}130786, NM_{—}130786.2, NP_{—}570602, NP_{—}570602.2; NM_{—}000014, NM_{—}00014.3, NP_{—}000005, NP_{—}000005.1; NM_{—}000662, NM_{—}000662.4, NP_{—}000653, NP_{—}000653.3; NM_{—}000015, NM_{—}000015.1, NP_{—}000006, and NP_{—}000006.1. Examples of Unigene IDs include, for example, those having formats Hs.529161, Hs212838, Hs.155956, Hs.2, Hs.534293, Hs.546822, Hs.506908, Hs.83347, Hs.315137, Hs.336768, Hs.429294, and Hs.421202. Examples of gene symbols include, for example, those having formats ADH6, ADH7, ADK, ADORA1, ADORA2A, ADORA2B, ADORA3, ADPRH, PARP1, ADRA1D, ADRA1B, ADRA2A, ADRB2, ADRB3, ADRBK1, ADRBK2, ADSL, ADSS, AP2A1, and AP2A2.

In one example, the user may input a search term. Once the user inputs a search term, a search or sorting feature is used to identify a sub-group of siRNA reagents from the collection that are matched to or related to the particular search term. The user may be presented with, for example, any number of matched siRNA reagents, for example the most closely matched siRNA reagent, or, for example the three, five, ten, fifteen, twenty, thirty, fifty, or one hundred most closely matched siRNA reagents to the search term. These may include, for example, the siRNA reagent most closely matched with the gene symbol, and may also include, for example, the siRNA reagents associated with genes involved upstream or downstream on the same cellular pathway as the gene associated with the gene symbol. The user may select at least one of the output siRNA reagents, and is then presented with matched biological reagents from the matched biological reagent collection. The siRNA reagents and the matched biological reagents may, for example, be presented with a purchasing function comprising links to the purchase of the siRNA reagents and matched biological reagents.

In another example, the user may order a custom-designed siRNA. Upon entering the order for the custom-designed siRNA, the user if provided with a collection of biological reagents matched to the siRNA target and any pathway or cellular process that is related to the target.

### Example 2

### Collections of Biological Reagents Comprising Isolated Proteins

Collections of biological reagents may comprise, for example, isolated proteins. Those of ordinary skill in the art will recognize that the present example relating to isolated proteins may be used to exemplify collections of biological reagents that comprise, for example, other biological reagents, such as nucleic acids and antibodies. Collections of biological reagents may comprise, for example, isolated proteins from any organism, including, for example, bacteria, insects, plants, and animals. Isolated proteins include, for example, isolated native proteins, isolated recombinant native proteins, and isolated recombinant proteins with post-translational modifications. Such collections may comprise, for example, mammalian isolated proteins, or, for example, humans isolated proteins, such as those presented herein in Table 13, or any of the isolated proteins disclosed at and linked to http address invitrogen.com on the date this patent application is filed, which collection is hereby incorporated by reference in its entirety. Such collections may comprise, for example, representatives of various protein families and classes, such as those presented herein in Table 12, wherein the proteins are arranged by protein functional family. Such protein functional families include, for example, proteins associated with biological processes, behavior, cell communication, cell-cell signaling, signal transduction, development, cell differentiation, embryonic development, growth, cell growth, morphogenesis, reproduction, physiological processes, cell death, cell homeostasis, cell proliferation, cell cycle, transport, ion transport, protein transport, death, metabolism, biosynthesis, protein biosynthesis, carbohydrate metabolism, catabolism, electron transport, energy pathways, lipid metabolism, DNA metabolism, transcription, protein metabolism, protein biosynthesis, protein modification, secondary metabolism, cellular component, cell, cell envelope, cell wall, intracellular, chromosome, nuclear chromosome, cytoplasm, cytoplasmic vesicle, cytoskeleton, cytosol, endoplasmic reticulum, endosome, Golgi apparatus, mitochondrion, peroxisome, ribosome, vacuole, lysosome, nucleus, nuclear chromosome, nuclear membrane, nucleolus, nucleoplasm, ribosome, nuclear membrane, plasma membrane, extracellular, extracellular matrix, extracellular space, unlocalized, molecular function, antioxidant activity, binding, calcium ion binding, carbohydrate binding, lipid binding, nucleic acid binding, DNA binding, chromatin binding, transcription factor activity, RNA binding, nucleotide binding, protein binding, actin binding, receptor binding, catalytic activity, hydrolase activity, nuclease activity, peptidase activity, kinase activity, protein kinase activity, transferase activity, enzyme regulator activity, motor activity, signal transducer activity, receptor activity, receptor binding, structural molecule activity, transporter activity, and ion channel activity.

A collection of biological reagents comprising isolated proteins may be searched, for example, by inputting a search term into an input interface or input function. Such search terms may include, for example, any term that may be used to identify the isolated protein, the nucleic acid or gene encoding the protein, biomolecules such as proteins that bind to or otherwise interact with the protein, the protein functional family of which the isolated protein is a member, the cellular pathway comprising the protein, or diseases associated with the protein or the cellular pathway. Search terms may include, for example, a gene symbol, accession number, FASTA header, key word, locus ID, Unigene ID, catalog number, protein name, biological pathway, disease, disease pathway, disease pathway member, cellular process, amino acid sequence, or a nucleotide sequence.

In one example, the user may input a search term. Once the user inputs a search term, a search or sorting feature is used to identify a sub-group of isolated proteins from the collection that are matched to or related to the particular search term. The user may be presented with, for example, any number of matched isolated proteins, for example the most closely matched isolated protein, or, for example the three, five, ten, fifteen, twenty, thirty, fifty, or one hundred most closely matched isolated proteins to the search term. These may include, for example, the isolated protein most closely matched with the gene symbol, and may also include, for example, the isolated proteins associated with genes involved upstream or downstream on the same cellular pathway as the gene that encodes the isolated protein. The user may select at least one of the output isolated proteins, and is then presented with matched biological reagents from the matched biological reagent collection. The isolated proteins and the matched biological reagents may, for example, be presented with a purchasing function comprising links to the purchase of the isolated proteins and matched biological reagents.

In another example, the user may order a custom-designed isolated protein. Upon entering the order for the custom-designed isolated protein, the user if provided with a collection of biological reagents matched to the isolated protein and any pathway or cellular process that is related to the isolated protein.

Protein Expression in Insect Cells

Isolated proteins may be isolated according to any method known to those of ordinary skill in the art, including, for example, isolating the native proteins from their native source, or isolating recombinant proteins by synthesizing them in vitro, or by isolating them from a recombinant source such as, for example, bacterial, plant, insect, or animal, such as mammalian, cells. Presented herein is an example of the expression and isolation of recombinant proteins from insect cells, although the isolated proteins of the matched biological reagent collections are not limited to those isolated from insect cells, or to the particular protocols presented herein.

Entry Clones Preparation and Plasmid Isolation:

E. coli cultures of human clones are inoculated into 2 ml deep well culture plates with 900 μl of 2× YT media containing 50 μg/μl Ampicillin and 50 μg/μl carbenicillin and incubated in a 37° C. floor shaker for overnight growth (220 rpm). The next day, plasmids containing hORF clones are isolated by Eppendorf's Perfectprep Plasmid 96 Vac Direct Bind kit (Eppendorf). Plasmid DNA is eluted with 70 μl of Molecular Biology Grade Water. Quality and quantity of DNA are visualized by running 5 μl of isolated plasmid DNA on a 1% E-Gel 96 agarose gel (Invitrogen).

LR Reaction into pDEST 20 Vectors:

The LR reaction is performed in a 10 μl volume in a 96-well PCR plate with the above entry clones and the destination vector pDEST20. 2.5 μl of the following mixture: 100 μl of LR reaction buffer (5× stock, Invitrogen), 50 μl of resuspended pDEST20 DNA (6 μg) and 100 μl of LR clonase (5× stock) is aliquoted into each well of a 96-well PCR plate, and 2.5 μl of the isolated entry clone plasmid is added into each well. The plate is sealed with an aluminum foil cover, spun down at 3000 rpm briefly and incubated at 25° C. overnight.

Transformation of pDEST20 LR into DH10Bac:

40 μl of DH10Bac competent cells are dispensed into each well of the 96-well plate containing the overnight LR mixture. A plate containing the cell mixture is incubated at 4° C. for 15 minutes, and then cells are heat-shocked at 42° C. for 40 seconds. After chilling, 120 μl of LB media are added to each well and the plate is incubated at 37° C. for 5 hours without shaking. At the end of the 5 hr incubation, 50 μl of cells are diluted into 500 μl of LB media containing Gentamycin (7 μg/l), Kanamycin (50 μg/μl) and Tetracycline (12 μg/μl) in a 2 ml 96 deep well culture plate. Cultures are incubated at 37° C. overnight (12-18 hrs) with shaking at 220 rpm. The next morning, the overnight culture is diluted into 800 μl of distilled water using a 96 pin replicator. 20 μl of diluted overnight culture from each well of the 96-well plate is plated onto one Nunc square plate containing LB media plus Gentamycin, Kanamycin and Tetracycline. Plates are incubated at 37° C. overnight. The next day, two Mantis 384-well output plates with 60 μl of LB plus Gentamycin (14 μg/μl) and Kanamycin (100 μg/μl) in each well are prepared, and 8 colonies from each transformation plate are picked into each well of the output plate by the Mantis colony picker. The output plates are incubated at 37° C. overnight.

Blue-White Colony QC:

Cultures in the output plate are replicated onto a LB/Bluo-Gal agar plate using a 384 pin replicator, and plates are incubated at 37° C. for at least 1 to 2 days or until the blue color developed. The blue and white colonies are analyzed using the Alpha FluorChem 8100. Wells which have nothing growing or have either light or blue colonies are failed for the next procedure. One passed colony from each clone is selected and rearrayed from the 384-well output plate into a 96-well 2 ml deep well plate containing 900 μl of 2× YT media plus Kanamycin 50 μg/μl and Gentarmycin 7 μg/μl.

Bacmid Isolation:

The culture plate is grown for approximately 20-22 hours at 37° C. with shaking at 180 rpm. The next day, bacmid DNA is isolated using Perfectprep BAC 96 kit following the manufacturer's protocol (Eppendorf). 5 μl of purified bacmid DNA is analyzed on a 1% E-Gel 96 agarose gel.

Transfection and Amplification:

Insect Sf9 cells are grown in SF-900 SFM medium supplemented with 10% (v/v) Fetal Bovine Serum (FBS) and 1% (v/v) penicillin/streptomycin, and incubated in a spinner flask at 26° C. with constant stirring at 100 rpm. On the day of transfection, cells are counted and diluted to a final cell concentration of 5×105 cells/ml in Grace's insect unsupplemented medium. 100 μl of cells are aliquoted into each well of a 96-well flat bottom tissue culture plate, and attached to the surface of the plate at 26° C. for 1 hour. Meanwhile, in a new 96-well PCR plate, the DNA and cellfectin mixture is prepared as follows:
Mixture A: 3 μl of Grace's insect medium is added into each well of a 96-well PCR plate first, then 3 μL of purified bacmid DNA from the above step is added to each well of the same plate to mix with the medium.
Mixture B: For each transfection, 0.3 μL of Cellfectin is diluted into 5 μl of Grace's insect unsupplemented medium.
   After adding mixture B to mixture A, the DNA:Cellfectin mixture is incubated at room temperature for 45 to 60 minutes. After 45 to 60 minutes of incubation time, for each transfection, 50 μl of Grace's insect medium is added to the mixture of A and B. Meanwhile, Sf9 cells are washed once with 100 μl of Grace's insect medium, and finally replaced with the diluted-mixture A and B (about 60 μl volume). Cells are incubated in 26° C. for 5 hours. After incubation, the supernatant which contains the transfection mixtures is removed, and is replaced with 100 μl of SF-900 SFM medium containing 10% FBS and 1% (v/v) penicillin/streptomycin. Cells are incubated at 26° C. for another 72 hours. At 72 hours posttransfection, the supernatant containing the original viruses (100 μl) is harvested and transferred into a sterile round-bottom 96-well plate. The plate is sealed and stored at 4° C. in the dark. For long term storage, viruses can be stored at (−80° C.). Original viruses are amplified once to increase the virus titer. 100 μl of Sf9 cells are plated out at 1×106 cells/ml density in each well of a 96-well tissue culture plate, and allowed to attach to the surface of the plate at 26° C. for at least half an hour. 2 μl of original virus are added to the cells, and cells are incubated at 26° C. for 72 hr. At 72 h post-infection, the amplified viruses are collected into a new sterile round bottom 96-well plate, can be stored at 4° C. or −80° C., or used directly for protein expression.

Protein Expression:

Sf9 cells are counted and diluted in SF-900 II SFM medium containing 10% FBS+1% penicillin/streptomycin to a final cell density of 2×106 cells/ml. 600 μl of Sf9 cells are aliquoted into each well of a 96-deep well cell culture plate, and 6 μl of the amplified viral stock are added to the wells. The plate is sealed with a Microporous sealing film which allows compressed air to permeate during incubation, and is loaded into the Higro™ cassette. The Higro™ is run at 26° C. with shaking at 450 rpm for 72 hours.

Protein Purification:

Boxes are lysed using a Harbil paint shaker for 30 seconds in 650 μL Tris lysis buffer with protease inhibitors, incubated shaking for 15 mins then lysed again for 30 secs. Lysates are clarified by centrifugation. 38 μL of glutathione-Sepharose 4B (GE Healthcare) is added, incubated at 6° C. for 1 hr with shaking, the slurries transferred to 96 well PVDF filter plates (Whatman) then washed twice with 200 μL of REPES wash buffer 1 and twice with 200 ul HEPES wash buffer 2. Proteins are eluted with 65 μL of Elution Buffer and consolidated into 384 well plates (Greiner, polypropylene/flat-bottom).

Western QC Sample Preparation:

At the end of expression period, 50 μl of cells from each well of the deep well culture plate are transferred into a new 96-well PCR plate. Cells are spun down, lysed in the lysis buffer and ready for further analysis as whole cell lysate. After proteins are purified, 10 μl of the purified protein is transferred into a new 96-well PCR plate. 10 μls of 2× SDS sample buffer are added to each well, and boiled in a PCR machine for 10 minutes.

SDS-PAGE:

The purchased precast gels are prerun at 150 volts for 30 minutes. Each gel has 26 lanes, therefore, 10 μls of the denatured purified proteins from two rows of the 96-well plate are loaded to the same gel using a 12-channel pipetman. On the same gel, 10 μl of the pertained protein molecular weight marker and the 10 μl of standard GST proteins (10 μg/μl) are loaded onto two separate lanes. Gels are run at a constant voltage of 150 volts for 1 hour or until the bromophenol blue marker dye is near the bottom end of the gel.

Blotting:

Each nitrocellulose membrane is labeled and soaked in the transfer buffer for a few minutes along with the Whitman 3 MM paper. The precast gel is opened, a nitrocellulose membrane is placed on top of the gel, and two Whatmann 3 MM paper are placed on each side of the gel-membrane. The gel sandwich is placed on the surface of the Semi Dry blotting apparatus with the nitrocellulose membrane on top of the gel. The electroblotting is performed at a constant current 250 mA for 20 minutes for each gel sandwich. After blotting, the apparatus is dissembled, and the membranes are probed immunochemically as described as follows:

Non-specific protein binding is blocked by incubating the membrane in blocking buffer (TBS, 0.5% Tween and 5% dried milk) for 2 hours at room temperature or overnight at 4° C.

Blocking buffer is discarded, and the membrane is incubated with primary antibody (Rabbit polyclone GST, 1:5000 dilution) in Blocking buffer for 1 to 2 hours at room temperature or overnight at 4° C.

Membrane is washed with Washing buffer for three times with 15 minutes of wash for each

Membrane is incubated with second antibody (1:5000 dilution for HRP conjugated goat antirabbit IgG) in TBS, 0.2% BSA for 1 to 2 hours at room temperature

Membrane is washed with washing buffer again for 3 times with 15 minutes of wash for each

Developing Membrane:

After the third wash of the membrane, it is ready for developing. Excess of washing buffer from the membrane is blocked by putting it on a paper tower for 5 seconds. A small piece of RADTape is placed on the side of prestained molecular weight marker on the membrane, the position of each band on the marker is manually marked on the tape. On a clean surface of a transparency sheet, 170 μl of solution A of the SuperSignal West Pico Maximum Signal substrate is mixed with 170 μl of solution B. The membrane is placed on top of this mixture, making sure it is covered by the solution completely. The membrane is scanned in the Alpha Innotech Fluoro Chem Apparatus, and the image is saved to a database.

Western QC Data Analysis:

The Western blot image is loaded into Western Kodak ID 3.5 software, and analyzed by the software. Based on the size of proteins on the molecular weight marker, the size of each band for each protein on the image is calculated by the software. All the data file is saved and uploaded into ProtoMine, and proteins are passed or failed Western QC based on the following criteria:
1. If the calculated molecular weight is within the 20% range of the predicted molecular weight, it is passed.
2. If the calculated molecular weight is above the 20% range of the predicted molecular weight, it is passed.
3. If the calculated molecular weight is below a 23% range of the predicted molecular weight, it is failed.
4. If a strong protein band is observed at the expected molecular weight for the GST tag, it is failed.
5. If no protein band is observed from Western blot, it is failed.

Concentration QC—The concentrations of human proteins are measured using microarrays. Human proteins and controls are printed on S&S FAST slides. The arrays are probed with anti-GST antibody followed by Alexa Fluor 647 antibody. The protein concentrations are derived from a GST standard gradient on the array and the spot intensities of the human proteins.

### Example 3

### Example of a Biomolecular Pathway Search

For purposes of illustration of a biomolecular pathway search, the calcium signaling pathway is used in the present example, the present invention is not limited to any particular pathway. Calcium (Ca²⁺) is a potent signaling molecule that is involved in many different cellular responses. Following receptor activation, members of the phosphatidylinositol-specific PLC (PI-PLC) family hydrolyze phosphatidylinositol 4,5 bisphosphate (PIP₂) to generate inositol 1,4,5 triphosphate (IP₃) and diacylglycerol (DAG). IP₃ initiates the release of intracellular Ca²⁺ from the endoplasmic reticulum. Extracellular Ca²⁺ influx is subsequently triggered through the activation of Ca²⁺ release activated Ca²⁺ channels (CRAC) by a process called capacitative Ca²⁺ entry.

Calmodulin, an intracellular Ca²⁺ sensor, binds to Ca²⁺ and activates the serine-threonine phosphatase calcineurin. Calcineurin dephosphorylates serine residues on the N-terminus of NFATc transcription factors activating nuclear translocation. In the nucleus, NFATc proteins bind to DNA in conjunction with other associated transcription factors (NFATn) to regulate gene expression.

Another protein family that is regulated by Ca²⁺ and DAG is protein kinase C (PKC). PKC is a serine-threonine kinase that regulates many different cellular processes including cell cycle, proliferation, differentiation, cytoskeletal organization, migration, and apoptosis. The PKC enzyme family includes three subgroups corresponding to conventional (α1 ,β1,β2,γ), novel (δ,ε,η,θ,μ), and atypical isoforms (ζ,λ). Although only the conventional PKC isoforms are activated by Ca²⁺, both the conventional and novel PKC isoforms are activated by DAG.

A user that requires products from a database related to the calcium signaling pathway may enter search terms related to this pathway. For example, the user may enter the term “calcium signaling pathway.” As a result of that search, the output may be, for example, all members of the biological reagent collection that match that pathway. For example, the output may include nucleic acids, proteins, siRNA reagents, antibodies, and cell lines expressing at least one of phosphatidylinositol-PLC, calmodulin, calcineurin, NFAT, and protein kinase C, as well as assay reagents such as phosphatidylinositol 4,5 bisphosphate (PIP₂), cell culture products, detection products, assay kits, enzymes, enzyme substrates, separation media, specific microarrays, and other matched biological reagents.

In one example, the user may first be presented with the name of the pathway members, wherein the pathway members are each linked to other matched biological reagents to that pathway member. Or, the user may input just one member of the pathway, for example, “calcineurin” and either obtain as output all the information and links for the pathway, or just members of the pathway and matched biological reagents related to calcineurin. In another search, the user may input the name of a disease in which the calcium signaling pathway is implicated, and receive as output the biological matched reagents for the pathway.

### Example 4

### Example of a Disease Pathway Search

For purposes of illustration of a disease pathway search, a search term related to a particular disease may be inputted. It is understood, however, that the present invention is not limited to any particular pathway. For example, upon inputting the keyword “Alzheimer's” in a search related to biological elements involved in a pathway implicated in Alzheimer's disease, the output may be, for example, all members of the biological reagent collection that match that pathway. For example, the output may include nucleic acids, proteins, siRNA reagents, antibodies, and cell lines expressing at least one of acyl carrier protein, acyl-ACP synthetase, ApoE2, ApoE1, ApoE3, BACE1, GGTase-1, Rac-1, Ras, Rab, Tau, and VEGF. The output may also include assay reagents such as cell culture products, detection products, assay kits, enzymes, enzyme substrates, separation media, specific microarrays, and other matched biological reagents. In one example, the user may first be presented with the name of the pathway members, wherein the pathway members are each linked to other matched biological reagents to that pathway member.

### Example 5

### Database Systems

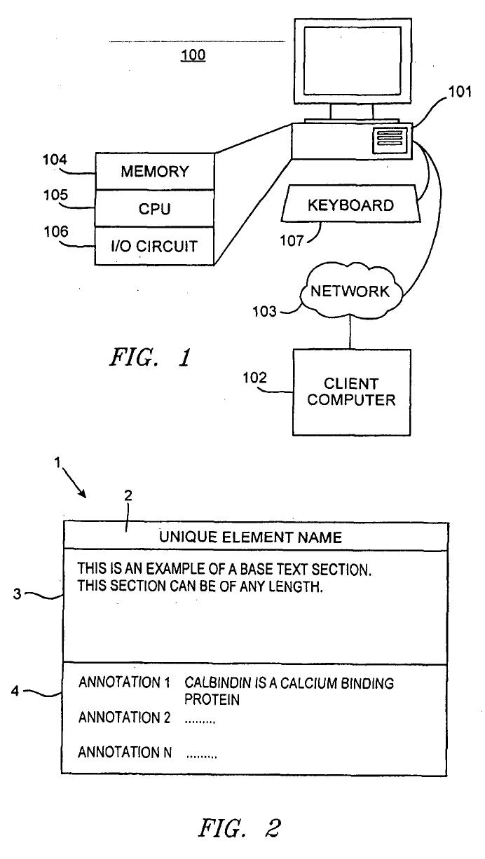

It will be appreciated by one of ordinary skill in the art that computer 101 can be part of a larger system (FIG. 1). For example, computer 101 can be a server computer that is in data communication with other computers. As illustrated in FIG. 1, computer 101 is in data communication with a client computer 102 via a network 103, such as a local area network (LAN) or the Internet.

In particular, computer 101 can include session tracking circuitry for performing session tracking from inbound source to net sale in accordance with the teachings of the present invention. In one embodiment, as will be appreciated by one of ordinary skill in the art, the present invention can be implemented in software executed by computer 101, which is a server computer in data communication with client computer 102 via network 103 (e.g., the software can be stored in memory 104 and executed on CPU 105), as further discussed below.

The present invention may be implemented using hardware, software or a combination thereof and may be implemented in a computer system or other processing system. An example computer system 100 is shown in FIG. 1. The computer system 100 includes one or more processors. A processor can be connected to a communication bus. Various software embodiments are described in terms of this example computer system. After reading this description, it will become apparent to a person skilled in the relevant art how to implement the invention using other computer systems and/or computer architectures.

Computer system 100 also includes a main memory, e.g., 104, preferably random access memory (RAM), and can also include a secondary memory. The secondary memory can include, for example, a hard disk drive and/or a removable storage drive, representing a floppy disk drive, a magnetic tape drive, an optical disk drive, memory card etc. The removable storage drive reads from and/or writes to a removable storage unit in a well-known manner. A removable storage unit includes, but is not limited to, a floppy disk, magnetic tape, optical disk, etc. which is read by and written to by, for example, a removable storage drive. As will be appreciated, the removable storage unit includes a computer usable storage medium having stored therein computer software and/or data.

In alternative embodiments, secondary memory may include other similar means for allowing computer programs or other instructions to be loaded into computer system 100. Such means can include, for example, a removable storage unit and an interface device. Examples of such can include a program cartridge and cartridge interface (such as that found in video game devices), a removable memory chip (such as an EPROM, or PROM) and associated socket, and other removable storage units and interfaces which allow software and data to be transferred from the removable storage unit to computer system 100.

Computer system 100 can also include a communications interface (106). Communications interface allows software and data to be transferred between computer system and external devices. Examples of communications interface can include a modem, a network interface (such as an Ethernet card), a communications port, a PCMCIA slot and card, etc. Software and data transferred via communications interface are in the form of signals, which can be electronic, electromagnetic, optical or other signals capable of being received by communications interface. These signals are provided to communications interface via a channel. This channel carries signals and can be implemented using wire or cable, fiber optics, a phone line, a cellular phone link, an RF link and other communications channels.

In this document, the term “electronic storage medium” is used to generally refer to media such as removable storage device, a hard disk installed in hard disk drive, and signals. These computer program products are means for providing software to computer system 100.

Computer programs (also called computer control logic) are stored in main memory and/or secondary memory. Computer programs can also be received via communications interface. Such computer programs, when executed, enable the computer system to perform the features of the present invention as discussed herein. In particular, the computer programs, when executed, enable the processor to perform the features of the present invention. Accordingly, such computer programs represent controllers of computer system 100.

In an embodiment where the invention is implemented using software, the software may be stored in a computer program product and loaded into computer system 100 using removable storage drive, hard drive or communications interface. The control logic (software), when executed by the processor, causes the processor to perform the functions of the invention as described herein.

In another embodiment, the invention is implemented primarily in hardware using, for example, hardware components such as application specific integrated circuits (ASICs). Implementation of the hardware state machine so as to perform the functions described herein will be apparent to persons skilled in the relevant art(s).

In yet another embodiment, the invention is implemented using a combination of both hardware and software. In addition, the data computer system preferably includes a display, which can be any device for displaying (101) information in a graphical form, a keyboard (107), which can be any device for inputting characters, and a mouse with a button, which can be any device for indicating screen position.

As envisaged by the present invention, the computer system possesses a database comprising matched biological reagent information of the present invention. In a related aspect, the choice of properties possessed by particular fields may include fields which are searchable and displayable or displayable only.

In a related aspect, the database is parsable. Parsing is the manner in which information is divided for searching. In a further related aspect, parsing may be viewed in at least one of two ways. One way is word-for-word (word parsing) where the computer breaks at every space. For example, with a title such as “The Electronic Mail Box,” the computer would break after “The,” “Electronic,” “Mail,” and “Box.” Thus, each word would be searchable. Further, with word parsing systems, the computer can be programmed to ignore words such as “the,” “of,” and, “but,” etc. Moreover, a hyphenated word may be read as a single word by the computer, so the text must be impeccably consistent if the system is to operate effectively.

A second method is phrase parsing. In this system, the breaks occur only where indicated “break.” The break indicator, or subfield delimiter, determines where each phrase is to be broken. Phrase parsing solves the problem of double-word descriptors. Within these breaks the information must be consistent in order to facilitate searching. Also, as envisaged by the present invention, a system can be programmed for both word and phrase parsing to make searching more extensive and complete.

Alternatively, a Boolean expression may be supplied by the user to retrieve files from the database (see, e.g., U.S. Pat. No. 4,384,325). For example, such an expression would involve a process of arithmetically comparing fields of records within a database to corresponding fields of records containing reference words in order to derive arithmetic, logical comparisons. The comparison results would be compared to inputs of a user supplied Boolean expression (e.g., those that contain AND, OR, AND NOT, etc.) to determine if the comparisons satisfy the user supplied Boolean expression. In one embodiment, there would be a corresponding indication where a Boolean expression hit is determined based on identification of an appropriate record and a separate indication as a Boolean expression miss whenever the Boolean expression is not satisfied upon determining the comparison.

The present invention may be embodied in a software program residing on a data processing system operating under Unix and/or Windows operating systems. In one embodiment, the software program is written in the perl, C, C++, C# and Java programming languages and uses the relational database management system, as the data storage.

According to the present invention, the data processing system receives a query, such as a natural language query, from a user and displays the terms of the query on a display screen. Each term is preferably displayed surrounded by a box. A displayed term and its surrounding box is called a “tile,” although the term “tile” should not be limited only to the use of a box surrounding a term. Instead, a “tile” refers more generally to a graphical representation corresponding to a displayed query term.

The data processing system, as envisaged, also preferably includes a dictionary and a thesaurus stored in another auxiliary memory, which is preferably an external hard disk drive, but could also be an external CD ROM or similar device. The dictionary contains a list of words that can be used, for example, as terms in the Boolean query and identifies the part of speech for each of the words. The words may be stored in the dictionary in “citation form,” which is a morphologically uninflected form that is related to a number of variations of the term. For example, the term “copy” may be preferably stored in the dictionary and identified as either a verb or a noun. The memory includes morphological rules to change words such as “copied,” “copies,” and “copying” to their citation form of “copy” before they are looked up in the dictionary. Similarly, certain query terms using lower case letters are stored in the dictionary with a citation form having all capital letters. Thus, “sql” would be stored as “SQL.” Such a system maintains a list of morphological rules for shortening words to their citation forms in memory and a list of parse rules for syntactic analysis in memory.

Target items and queries may be associated with tags as flags for generating and sending notices, such as a single flag to trigger notification of non-user managers/systems (e.g., sales, manufacturing, news release, IT maintenance and security, accounting, financial management or support etc.). In a related aspect, multi-flag notices are envisaged, where a set of flags is associated with target items or queries, which then trigger such notification as above. In a further related aspect, override flags such as not to notify a security function when for example, the query is from a specific source or list of sources. In another related aspect, the multi-flag tagging involves the use of a decision tree to determine which if any of the non-user managers/systems are to be notified.

A thesaurus stores lists of words related to citation terms. The related words preferably include more specialized/more general words, lists of synonyms, alternative terms and lists of related terms. The exact organization of both the dictionary and thesaurus is not important to the present invention. Any organization that will accommodate the invention may be used.

In a related aspect, most files, such as those produced by the large time-sharing vendors, have what is known as a “basic index,” or “default file.” This file index consists of the basic controlled term vocabulary as well as terms preceded by their categorical mnemonics, such as OR for “organism,” NA for “nucleotide accession,” GN for “gene name,” or RF for “references.” In one embodiment, searching can be processed using the mnemonic tags or codes or through general, or natural language terms. In one embodiment, for each index an inverted file is created. The advantage of an inverted file is its speed.

In one embodiment, the database comprises sets of named annotated text strings. Each element of the set is defined (e.g., unique identification, base text, etc.). Annotations can be applied to any element of the set (e.g., base text).

An example of data set entry is illustrated in FIG. 2. The entry 1 comprises a unique element (identification) name 2, a base text section 3, and an annotation section 4.

In another embodiment, further additional indexing may be attached. For example, providing full-text searching in addition to a basic index. Such a full-text search increases the coverage of the search. In a related aspect, the search can be absolutely scoped (limited to only certain parts of a site) or scoped to a topic, category or idea.

“Dialog box” refers to sub-widows that open to provide a user with a set of options from which to choose. The dialog box may contain control options that are split into two or more tabs. Tabs may include, but are not limited to Search By Sequence, Search By Keyword/ID, Browse By Ontology and ORF FAQs (Frequently Asked Questions). Further, the dialog box may contain one or more buttons that present the user with two or more mutually exclusive options. For example, to limit search to human or mouse species for a sequence search, a user may check the appropriate button in the dialog box prior to search.

Right-clicking and shortcut menus are available, to get quick hints about what an item is or what it can do to view its shortcut menu. The short cut menu can offer a list of options e.g., properties, printing, open a new window, save target as, add to favorites, define how item functions and/or proper method of interfacing by user.

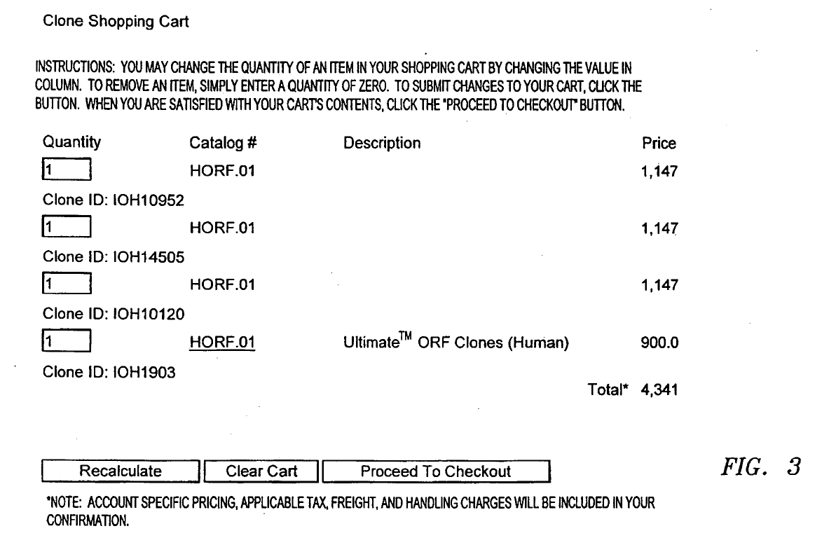

The user interacts with the system through a user interface. A user interface is something which bridges the gap between a user who seeks to control a device and the software and/or hardware that actually controls that device. The user interface for a computer is typically a software program running on the computer's central processing unit which responds to certain user-entered commands. Order entry system (FIG. 3) uses object-based windows as the preferred user interface. In a related aspect, PowerBuilder® by Powersoft Corporation is used as the window development tool.

In one embodiment, the present invention can be implemented using an interactive graphical user interface for specifying and refining database queries. One example of such an interface is provided by the “AVS™” visual application development environment manufactured by Advanced Visual System, Inc., of Waltham Mass. Another example of a visual programming development environment is the IBM® Data Explorer, manufactured by International Business MAchines, Inc. of Armonk, N.Y.

It is noted that using a visual-programming environment, such as AVS, is just one example of a means for implementing an embodiment of the present invention. Many other programming environments can be used to implement alternate embodiments of the present invention, including customized code using any computer language available. Accordingly, the use of the AVS programming environment should not be construed to limit the scope and breadth of the present invention.

In one embodiment, using such a system reduces custom programming requirements and speeds up development cycles. In addition, the visual programming tools provided by the AVS system facilitate the formulation of database queries by researchers who are not necessarily knowledgable about databases and programming languages. In addition, an advantage to using a programming environment such as AVS, is that the system automatically manages the flow of data, module execution, and any temporary data file and storage requirements that may be necessary to implement requested database queries.

AVS is particularly useful because it provides a user interface that is easy to use. To perform a database query, users construct a “network” by interacting with and connecting graphical representations of execution modules. Execution modules are either provided by AVS or are custom modules that are constructed by skilled computer programmers. For example, customized AVS modules can be constructed using a high level programming language, such as C, C++ or FORTRAN, in accordance with the principles as described.

The purpose of constructing a network in AVS is to provide a data processing pipeline in which the output of one module can become the input of another. In one aspect of the present invention, database queries are formulated in this manner. A component of the AVS system referred to as the “Flow Executive” automatically manages the execution timing of the modules. The Flow Executive supervises data flow between modules and keeps track of where data is to be sent. Modules are executed only when all of the required input values have been computed.

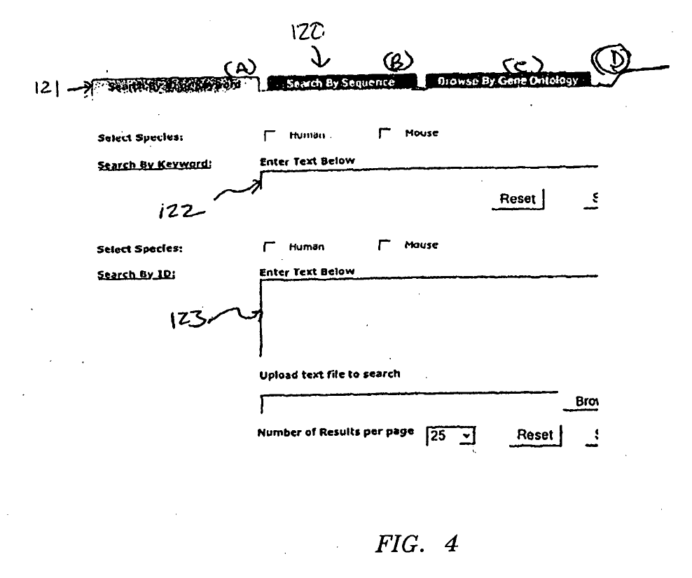

One envisaged user interface is shown in FIG. 4. The user interface employs window 120 preferably in the form of a rectangular shaped box having a toolbar 121 across the top which provides a set of standard menu options represented by a plurality of tabs or buttons A through D.

Window 120 also includes a plurality of other tabs/buttons represented preferably as search options. Tab A typically represent an action or choice, which is activated immediately upon user selection thereof. The tabs/buttons on window 120 may contain text, graphics or both. In a related aspect, buttons A through D contain graphics (i.e., icons) so that the user may readily determine the function they represent.

Window 120 preferably includes a plurality of data capture fields 122 and 123 for capturing data. The data capture fields allow the capture of variable length text. The data can be captured either automatically by system-to-system communication or by the user, such as through a keyboard.

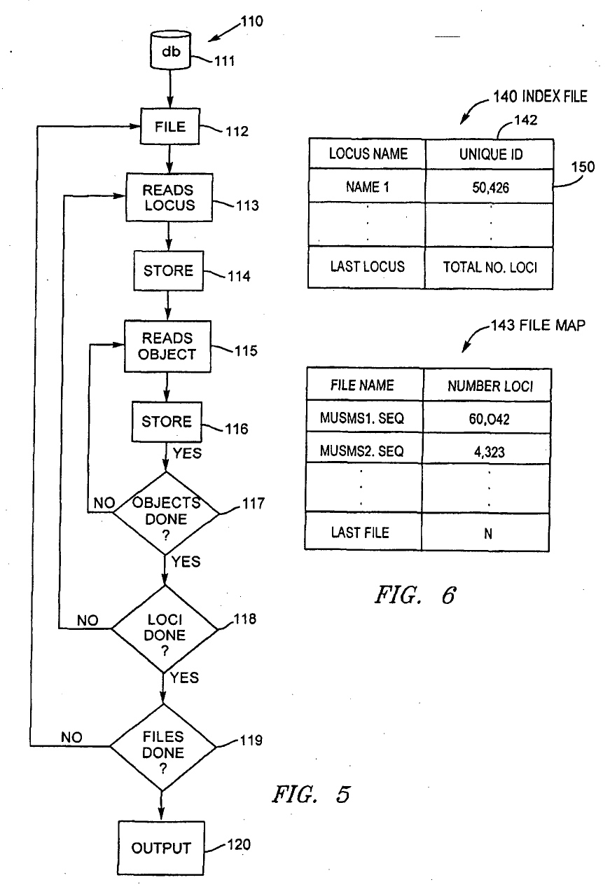

FIG. 5 is a flowchart (110) that depicts the beginning process that can be used to search for a record. The process begins with step 111, where control immediately passes to step 112. In step 112, the process opens the next ORF file. Typically, the first time step 112 is executed, the first file listed in the file map is opened. An example of a file map can be seen in FIG. 6. FIG. 6 illustrates in block diagram form the contents of an index file and a file map in accordance with an embodiment of the present invention.

As shown, the index file 140 comprises, for example, the unique Name 1 of each element in the database (see e.g., FIG. 2), and a unique ID 142 that is assigned to each element. Typically, the unique ID 142 assigned is simply the order number in which the entry appears in the database. Typically, when multiple files are used, their ordering is performed according to the file map described below.

A file map 143 may comprise the file name of each file in the database, and the number of entries (loci) within each file. Thus, given a loci number (i.e., the unique ID 142 assigned to each loci, as described above), one can easily determine which file contains the entry by consulting the file map 143.

Returning to FIG. 5, next, in step 113, the process parses the file and reads the next locus in the file. Of course, the first time step 113 is executed for each file, the first locus in the file is read. Next, as indicated by step 114, the offset and length of the locus read and parsed in step 113 is stored in an associated card file (card files contain a road map pertaining to the searchable objects within the associated locus). Typically, for example, the card file would have same name as the associated sequence file for identification purposes. For example, for a mouse file named “MUSMS.SEQ,” the associated card file is named “MUSMS.CRD.”

Next, as indicated by step 115, the next searchable object is read. For example, the first time this step is executed, the LOCUS section is read and its offset and length are determined. This offset and length is next stored in the associated objects file, as indicated by step 116. Typically, for example, the objects file would have the same file name (but different file type), as the associated sequence file for identification purposes. For example, for a mouse file named “MUSMS.SEQ,” the associated parameter file is named “MUSMS.OBJTS.”

Next, as indicated by step 117, the process determines if there are additional searchable objects in the locus. If so, control loops back and steps 115 and 116 are executed, thereby storing offsets and lengths for all searchable objects in the locus, until all searchable objects have been processed.

As indicated by step 117, once all searchable objects have been processed, control passes to step 118. In step 118, the process determines if there are any additional loci remaining in the file read in step 117. If so, control passes back to step 113, and the next locus is processed in the same manner as described above. Once the last locus in the file has been processed, control passes to step 119, as indicated.

In step 119, the process determines if there are any more files listed in the file map that need to be processed. If so, control passes back to step 112, where the next file is opened. Next, the process repeats itself, as described above, until all files have been processed in the manner described above. Finally, as indicated the process ends with step 120.

The net result of the process depicted in FIG. 5, is the creation of an index file and an objects file (i.e., extract) for each file used in a particular implementation of the present invention.

The index files and object files are each read into memory and a file name is associated for each Unique ID once the system receives a request to perform a search on a particular locus.

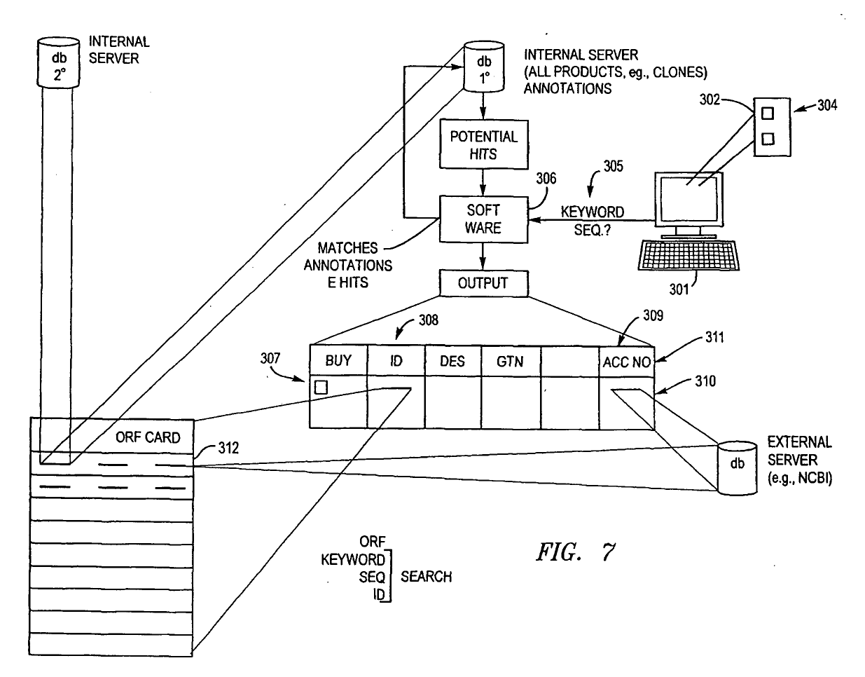

A flow chart for use of the index file and object file is shown in FIG. 7. A user interface 301 allows the user to input parsable/searchable information (e.g., a word, phrase, sequence, ID number). Optionally, the search can be scoped by activating GUI 304 prior to inputting parsable/searchable information 305. In the next step, the scoped search limits access to only a certain portion of all of the products available on the database 302 (e.g., all mouse data, each associated with a unique ID). Software 306 processes the inputted command to limit output to only those files matching the keyword within the scoped products, e.g., page 311.

The output page will contain a list of hits 307 corresponding to the input command, where the user can point to embedded hyperlinks to access annotation data associated with, for example, a unique ID number 308 or accession number 309. If the hyperlink for the unique ID number 310 is activated, the number is used to search the index file and the corresponding data is matched to the objects file. Matching of the index and object file will retrieve the appropriate locus from the ORF file database 312 and an annotated document for the unique ID number will be displayed to the user.

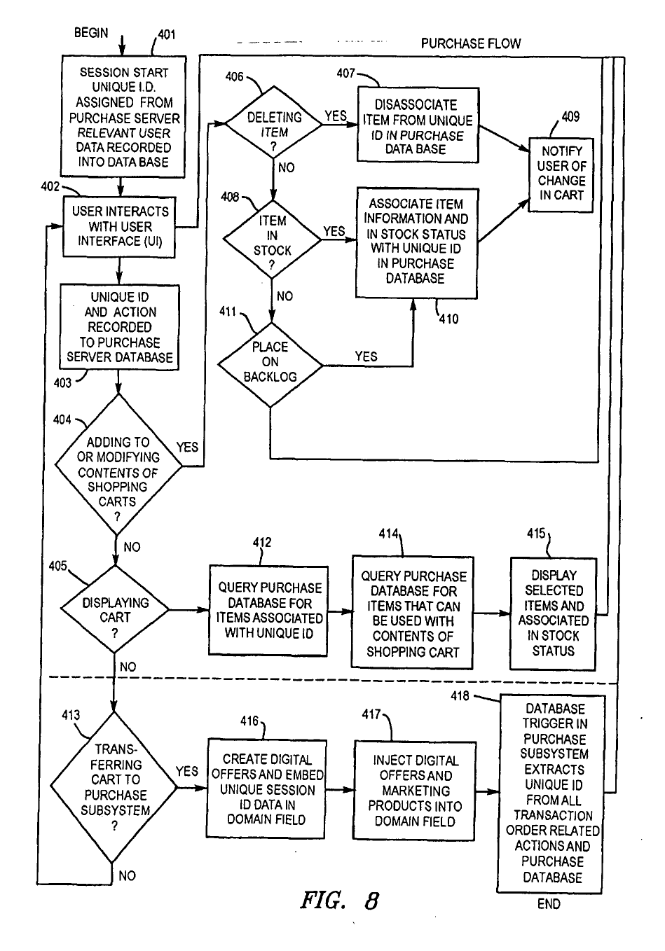

FIG. 8 is a purchase flow diagram of interactive network session tracking from inbound source to net sale in accordance with one embodiment of the present invention. Operation begins at stage 401 in response to a new user initiating access to an interactive network site. At stage 401, a unique session ID (identifier) is assigned from a front-end session database, and relevant user data is recorded in the session database associated with the session ID. For example, the relevant user data includes the user's inbound source (origin), such as a unique source ID of a banner (advertisement) on a search engine WWW site (e.g., which can be determined using standard name-value pairs passed via HTTP protocol).

At stage 402, the user interacts with the user interface of the network site. For example, the user interacts with the WWW online site by adding or deleting items from a virtual shopping cart or by jumping to different, dynamically generated HTML pages of the WWW site. At stage 403, any action performed by the user during stage 402 is recorded in the session database and associated with the session ID.

At stage 404, whether the user added or modified items in the shopping cart during stage 402 is determined. If so, operation proceeds to stage 406. Otherwise, operation proceeds to stage 405. At stage 406, whether an item is to be deleted from the shopping cart is determined. If so, operation proceeds to stage 407. Otherwise, operation proceeds to stage 408. At stage 407, the deleted item is disassociated from the session ID in a purchase server shopping cart database. Operation then proceeds to stage 409, which is discussed below. At stage 408, whether the item to be added is in stock is determined. If so, operation proceeds to stage 410. Otherwise, operation proceeds to stage 411. At stage 410, the added item is associated with the session ID in the shopping cart database. The in-stock status is also associated with the session ID in the shopping cart database. At stage 411, the out-of-stock item is placed on backorder. The entry in the shopping cart database that is associated with the session ID is then appropriately updated at stage 409. At stage 409, the user is notified of the change in the shopping cart. For example, the user is appropriately notified of the added or modified item(s) in the shopping cart.

In one embodiment, if the item is out of stock or the item requires custom service (e.g., but not limited to, antibody generation, clone production, vector design, nucleic acid/primer design, etc.), alternatively, the user can be linked to a product service page for such custom service. Further, the user can be linked directly to a service, technical or customer representative.

At stage 405, whether the user desires to have the contents of the user's shopping cart displayed is determined. For example, the user may want to view the currently added items in the user's shopping cart. If so, operation proceeds to stage 412. Otherwise, operation proceeds to stage 413. At stage 412, the shopping cart database is queried for items associated with the user's session ID. This can include items or services that can be used in connection with contents of the shopping cart (e.g., enzymes, clones, vectors, antibodies that can be used with protein query, custom designs for plasmids, maps, host organisms, etc.). At stage 415, the selected items and associated in-stock status are displayed to the user. For example, the user's selected items for purchase are output to the user's display.

At stage 413, whether the user is ready to purchase the currently selected items is determined. If so, operation proceeds to stage 416 and transitions to a (secure) purchase subsystem (e.g., a purchase subsystem that communicates via the Internet using an encrypted protocol to protect sensitive financial data). Otherwise, operation returns to stage 402. In particular, as shown by the horizontal dashed line of FIG. 8, if the user elects to proceed to purchases of the selected items in the user's shopping cart, then operation transitions across a seam between a first subsystem and a second subsystem of the network site (e.g., a WWW server). In one embodiment, the first subsystem is a catalog subsystem, which uses standard HTTP protocol, and the second subsystem is a secure purchase subsystem, which uses standard SSL (Secure Sockets Layer) protocol (i.e., an encrypted protocol for security purposes).

At stage 417, a digital offer is created to execute a net sale transaction (e.g., a customer order) of the selected items. For example, the shopping cart data stored in the shopping cart database can be passed to Open Market's commercially available TRANSACT software for creation of one or more digital offers (e.g., one digital offer per product). The session ID is embedded in the Domain field (also called the unique ID field) of each digital offer such that inbound source, user activity at the network site, and net sales data are all associated with the same unique session ID for subsequent (e.g., offline) correlation and analysis.

At stage 418, the digital offer is injected into a transaction database, such as the commercially available Open Market TRANSACT database. Thus, the user's shopping cart data is also maintained in the transaction database of the purchase subsystem and is associated with the user's unique session ID.

The user can modify items in the user's shopping cart after entering into the purchase subsystem. For example, the user may decide to delete an item from the user's shopping cart. Accordingly, at stage 418, the shopping cart data associated with the session ID that is stored in the Open Market TRANSACT database is extracted from all TRANSACT order-related actions and the shopping cart database is appropriately updated. Accordingly, the shopping cart database of the catalog subsystem is synchronized with the shopping cart data stored in the transaction database of the purchase subsystem. If the user executes any further interactions with the user interface of the WWW online site, then operation returns to stage 402. Otherwise, (i.e., the user exits the browser session) operation terminates.

In a related aspect, each new record includes the new session ID, a source ID (i.e., an inbound source), a time stamp, a referrer URL (Universal Resource Locator), an IP (Internet Protocol) address, and an entry point (e.g., WWW online site start page). The session ID is associated with the user's browser session using a standard transient (HTTP) cookie (i.e., the cookie stored on the user's computer includes the session ID). Thus, the user's subsequent actions (e.g., HTTP requests) are associated with the user's unique session ID at least until the user exits the user's browser (i.e., the users session is viewed as the life of the user's browser session).

In one embodiment, such user information can be used to track the accumulation of materials for illicit purposes (e.g., bio-terrorism), where orders to be shipped to separate sites for assembly may be tracked back to the same URL.

In another related aspect, every WWW page (e.g., HTML page) that is viewed is tracked in the session database and associated with the session ID. Further, every shopping-cart-related activity is tracked in the session database and associated with the session ID. In particular, the session database records include the following: the session ID, the time stamp, the page viewed or nature of interaction, and (for shopping-cart-related activities) the online products or services added or modified.

In a further related aspect, when adding a product to the shopping cart, a new record is added in the shopping cart database. For example, the new record includes the session ID, a model identifier, an in-stock indicator (e.g., Y or N for in stock or out-of-stock, respectively, which can then be interpreted to determine if an added item is on back-order), and a quantity. Moreover, when modifying the quantity of an item already in the shopping cart, the record in the shopping cart database containing the item is located using the session ID, model, and in-stock indicator as criteria. The appropriate criteria can then be updated. An adjusted quantity can trigger a change to an out-of-stock indicator if the quantity exceeds available inventory. At stage 406, when deleting a product from the shopping cart, the appropriate record is located as similarly discussed above. The located record can then be deleted.

### Example 6

### Advanced Search Modules

The present Example provides an illustration of advanced search modules that may be used to search a biological element and obtain matched biological reagents. Such search modules may be designed such that the output includes matched biological reagents, or, for example, the initial output on the first page would include only the specific target molecules that are the results of the search, each comprising a hyperlink to matched biological reagents to that target molecule.

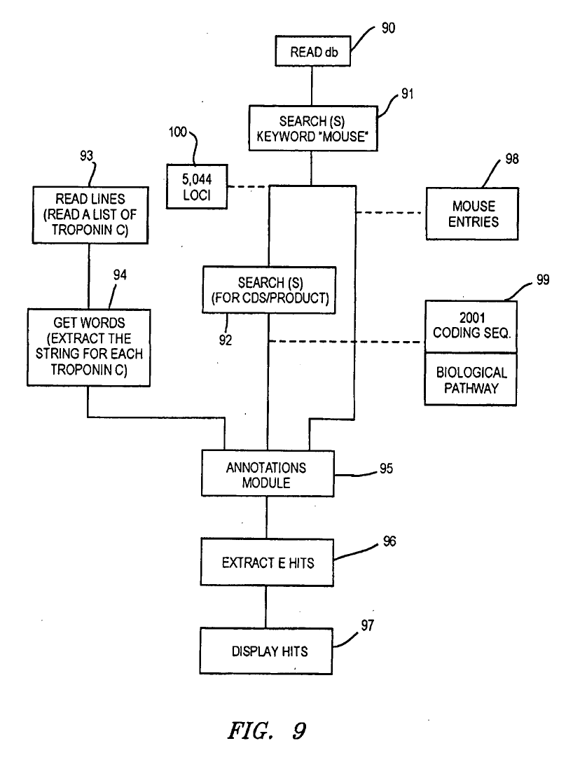

Advanced search modules 120 identify the way in which a user may retrieve objects from the server for that are of procurement interest. A dialog flow for the advanced search modules is shown in FIG. 9.

In FIG. 9 a search is performed in the mouse database to search for troponin C for mice. As shown, the first step is to execute the read database module 90. The output is the mouse portion of the database. Next, as indicated, the search database module 91 is executed. In this case, the user enters search parameters to extract all “mus musculus” (mouse) entries from the database. As indicated by the output block 98, this results in a total of 60,055 entries.

Next, the search database module 92 is again executed. This time the input is the 5,044 mouse loci from module 81. This time the search is performed to find coding sequences (CDS). A read lines module 93 is executed in parallel for reading in a pre-compiled list of named troponin c sequences. Next, as indicated, a get-words module is used to extract the sequence from each of the named troponin C sequences.

Next, the search database module 95 is executed. The search database module 95 has three input parameters. The first input parameter is the Hits list 100 comprising the 5,044 mouse loci. The second parameter is the Hits list 99 comprising the 2001 coding sequences. The coding sequences 99 are used to provide a context to the Annotation module 95. This annotation is used in conjunction with parameters from the vendor that defines the relationship for the annotation. For example, the vendor can specify a search for troponin c sequence 93 that is associated with pathway information 99

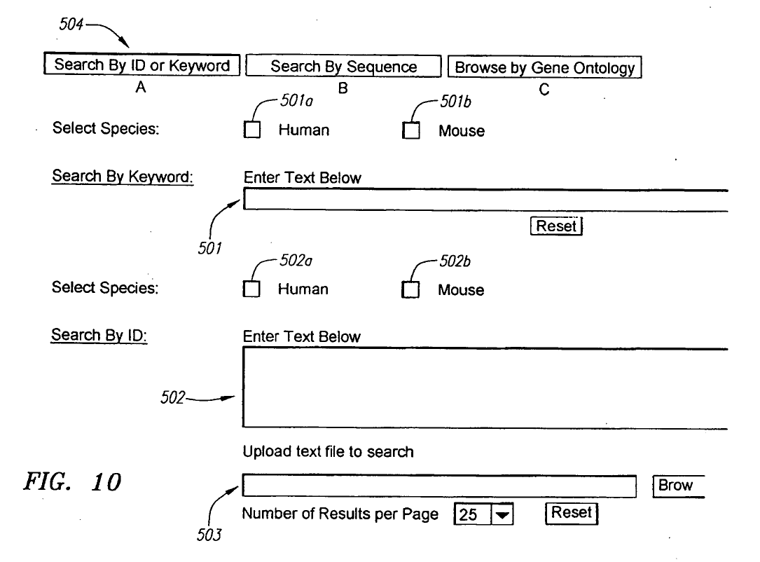

In order to initiate a search, the user must be able to pull up a subset of target items from the system. In this regard, the advanced search modules used are made up of at least 3 functions (FIG. 10), namely Search By Keyword/I.D. (which includes text file searching), Search By Sequence, and Browse By Ontology, all of which may be further parsed by selection of species (501(a) and (b)). These functions may be represented by tabs 504(A), (B), and (C) of the user interface of FIG. 10. For example, such dialog boxes may include Search By Keyword (to include Select Species buttons 501(a) and (b)) 501, Search By ID (to include Select species buttons) 502, and Upload text file to search 503.

Search By Keyword

Prior to activation of Search By Keyword 504, buttons are available for selection of species (501(a) and (b)). Further, the number of results per page can be delimited on the first page of the browser.

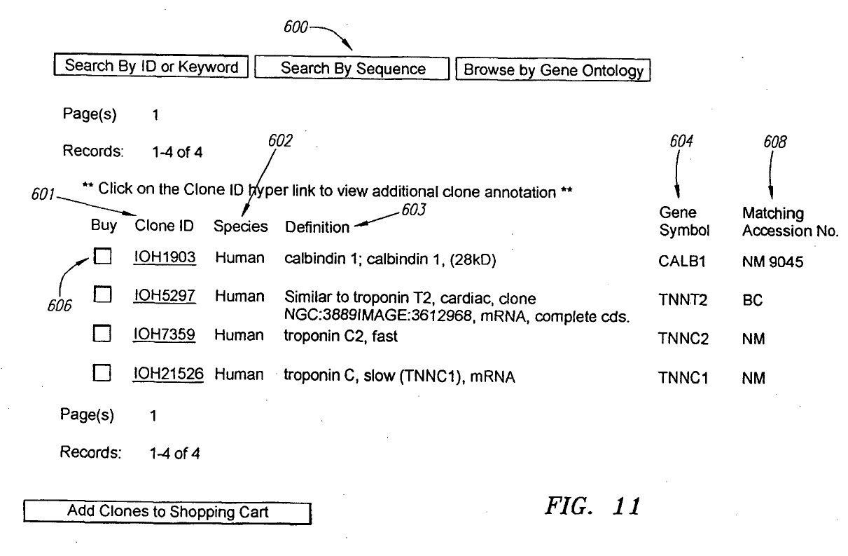

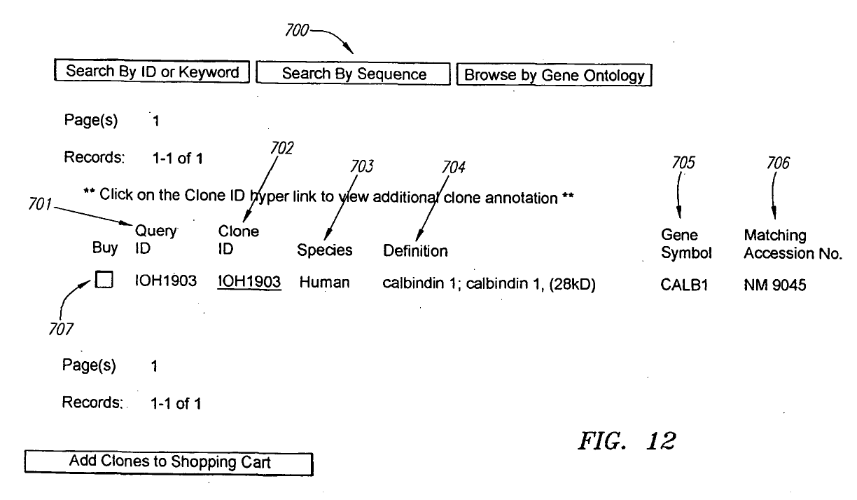

Upon inputting of keywords in the appropriate dialog box, a window 600 as shown in FIG. 11 opens and permits the user to view the products which conform to the biological attributes associated with the keywords. The search results window 600 defines the number of pages and records which conform to the search criteria of the user. As is shown from search results window 600 of FIG. 11, 5 search criteria data fields are preferably identified. These include a Clone ID field 601, species field 602, definition field 603, Gene Symbol filed 604 and Accession Number field 605. Also included is a button for the option to buy the biological material(s) meeting the criteria of the search (606).

It is understood that the search criteria will vary depending upon the keywords and species selected. Upon selecting a keyword and species, window 600 displays at least one page of results representing a number of records associated with the keywords currently used. For example, in the case of troponin C (human), window 600 provides results page displaying the number of pages encompassing the records, the number of records, option to buy, Clone ID, Species, Definition of the clone, Gene Symbol and Accession Number associated with the cloned gene (FIG. 11).

Search by ID

Prior to activation of Search By ID 502, buttons are available for selection of species (502(a) and (b)). Upon inputting of appropriate ID (e.g., Catalog Number(s), GenBank Accession(s) Gene Symbols(s), LocusLink ID(s), Unigene Cluster ID(s), etc.) in the appropriate dialog box, a window 700 as shown in FIG. 12 opens and permits the user to view the products which conform to the biological attributes associated with the ID numbers. The search results window 700 defines the number of pages and records that conform to the search criteria of the user. As is shown from search results window 700 of FIG. 12, 6 search criteria data fields are preferably identified. These include a Query ID field 701, Clone ID field 702, species field 703, definition field 704, Gene Symbol filed 705 and Accession Number field 706. Also included is a button for the option to buy the biological material(s) meeting the criteria of the search (707).

Again, it is understood that the search criteria will vary depending upon the type of ID used and species selected. Moreover, text files can be uploaded from the users computer to the browser page at the “Upload Text File to Search” field for subsequent search (FIG. 10, 503).

Search by Sequence

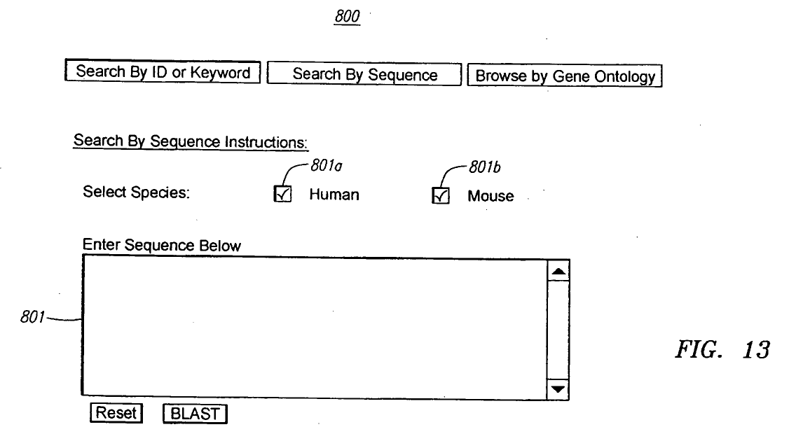

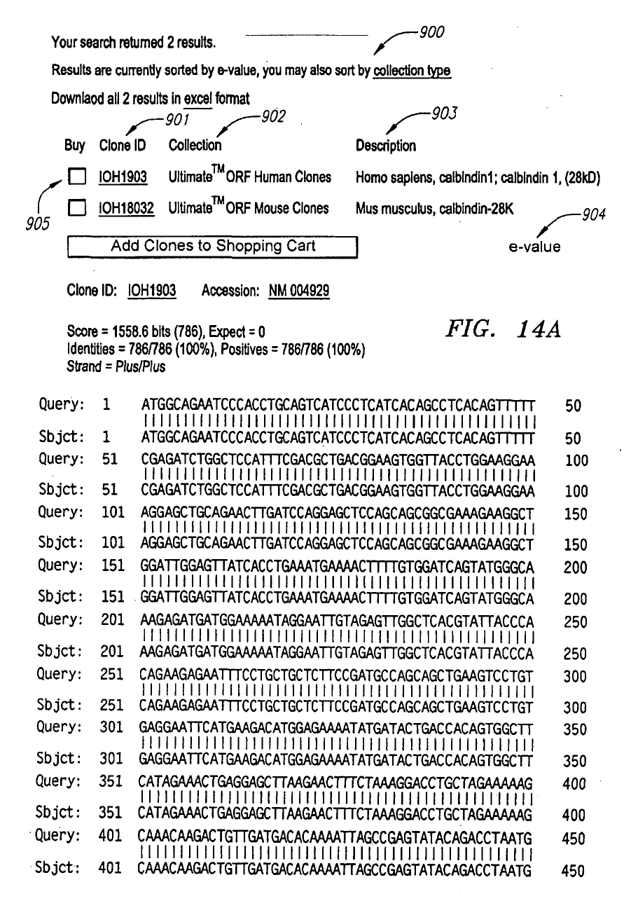

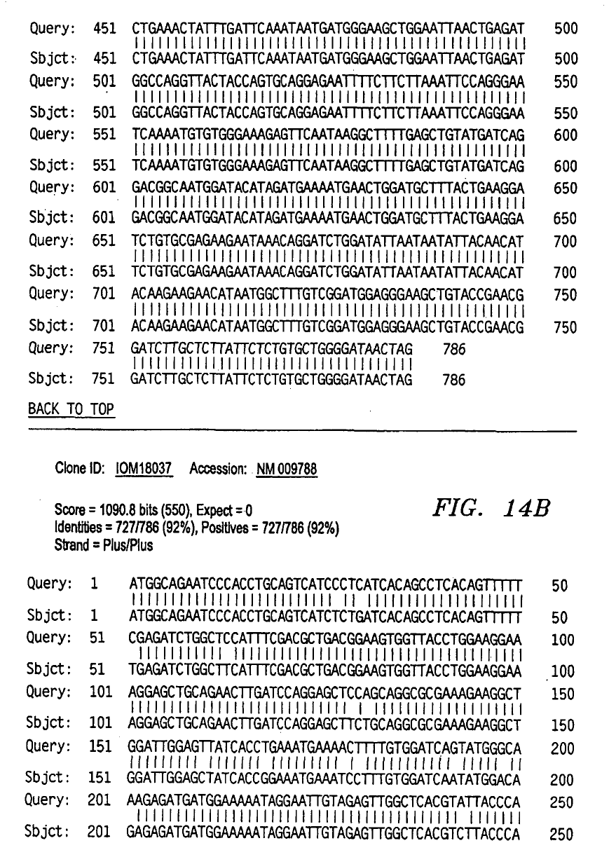

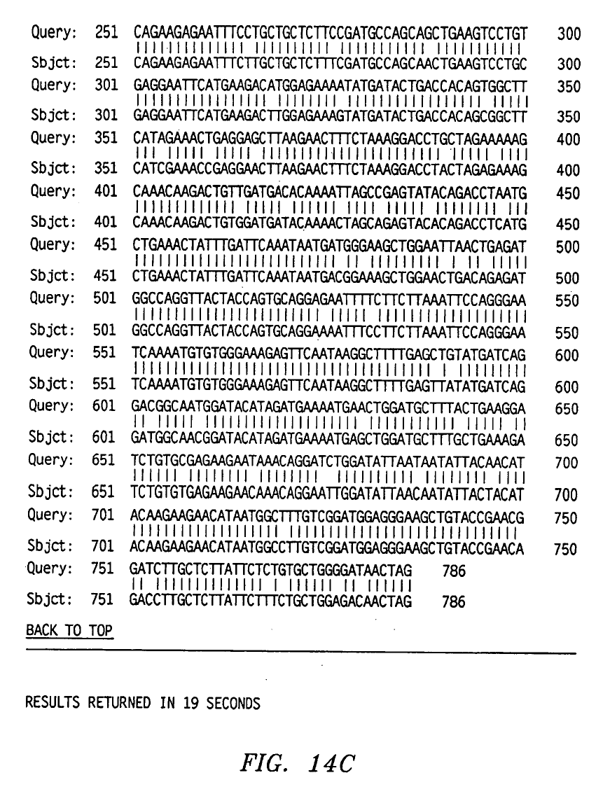

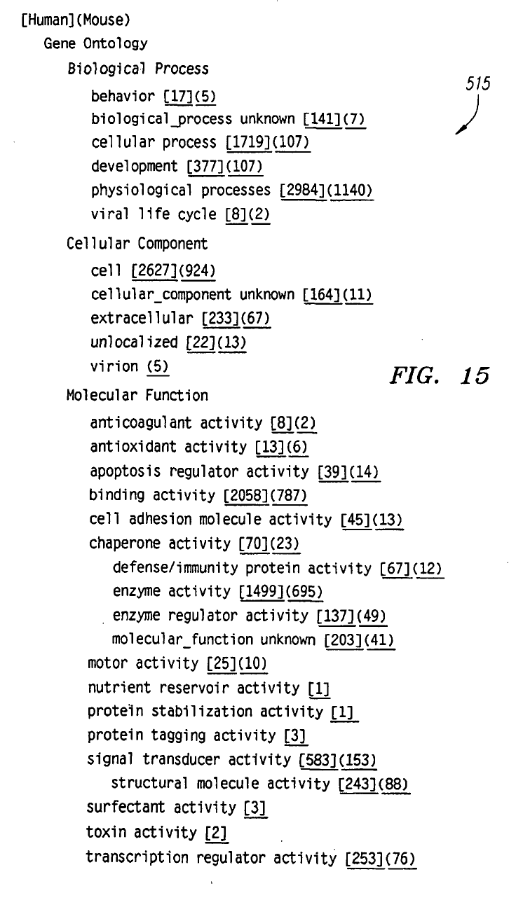

Prior to activation of Search By Sequence, buttons are available for selection of species (FIG. 13, 801(a) and (b)). Upon inputting of appropriate sequence (e.g., the input sequence window accepts nucleotide/amino acid sequences between 50 and 10,000 residues in FASTA, GenBank, and text formats, blastn is used to search the clone databases and results with e-values less than 0.01 are reported, etc.) in the appropriate dialog box (801), a window 900 as shown in FIG. 14 opens and permits the user to view the products which conform to the biological attributes associated with the sequence. The search results window 900 defines the number of results which conform to the search criteria of the user. As is shown from search results window 900, 4 search criteria data fields are preferably identified. These include a Clone ID field 901, collection field 902, description field 903, and e value 904. Further a field is available for linking user to the specific sequence described in 904. Also included is a button for the option to buy the biological material(s) meeting the criteria of the search (905).

Browse by Ontology

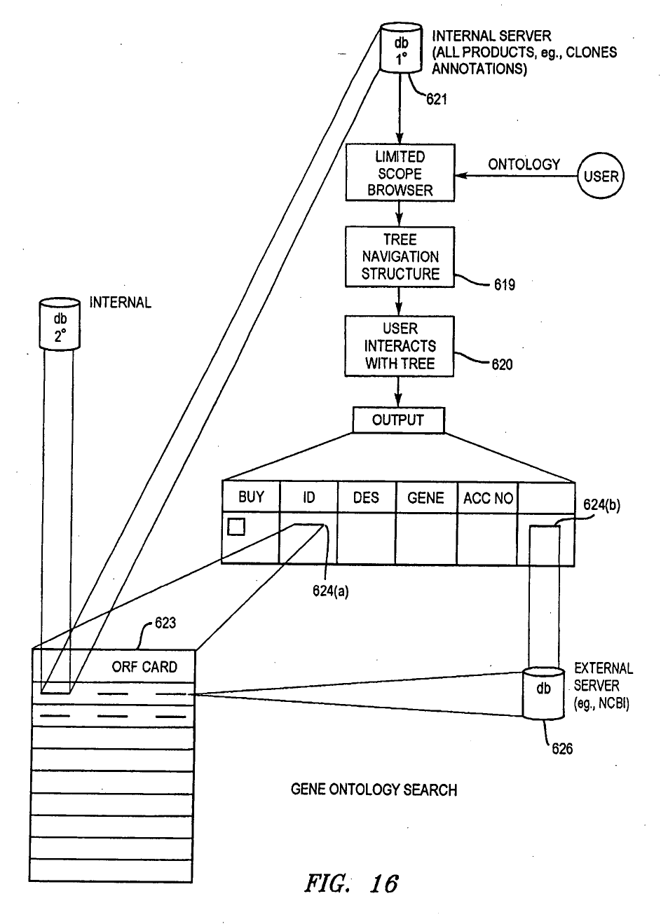

Activation of the Browse by Ontology tab triggers a keyword jump which loads a separate limited scope page (FIG. 15, 115). The illustration in FIG. 16, diagrams the flow (116). Using tree navigation (119), the gene ontology page displays, for example, three categories for viewing/activation by the user (e.g., Biological Process, Cellular Component, or Molecular Function). The user then activates a GUI (e.g., button, 120), that displays a number of headings (behavior, biological process unknown, cellular process, development, obsolete, physiological processes, viral life cycle, etc.) within that category. Optional indicators may include, but are not limited to, the number of subcategories under each category. The headings are followed by selectable species designations (e.g., human, mouse, etc.), which the user can activate, resulting in a search results window as described above.

The search results windows also contains hyperlinks (124(a) and (b)) which may lead to another WWW site (126), or another place within the same browser (121). In the exemplified system, after a clone has been selected, the user can click the hyperlink in the Clone ID field (124(a)) which leads to an electronic (ORF) card for the selected clone (123). The card may contain headings such as gene information, open reading frame (ORF) information, clone information, protein information, single nucleotide polymorphism information, and genomic-links. In a preferred system, the headings are followed by fields containing hyperlinks to both commercial and private databases (e.g., gov't, universities, consortiums, etc. (126)) which provide further information regarding the category as denoted by the heading.

The Ontology database is regularly updated by manual inputting of new data or by tracking using a Web robot to search the World Wide Web for such new data (e.g., see U.S. Pat. No. 6,718,363).

In one aspect, a preference database may be generated to contain profile data on a user. In a related aspect, a type of device for building a preference database is a passive one from the standpoint of the user. The user merely makes choices (e.g., menu choice in a browser built into a reader) in the normal fashion and the system gradually builds a personal preference database by extracting a model of the user's behavior from the choices. It then uses the model to make predictions about what products or services the user would prefer in the future or draws inferences to classify the user (e.g., an industrial scientist or an academic scientist). This extraction process can follow simple algorithms, such as identifying apparent preferences by detecting repeated requests for the same product or service, or it can be a sophisticated machine-learning process such as a decision-tree technique with a large number of inputs (degrees of freedom). Such models, generally speaking, look for patterns in the user's interaction behavior (i.e., interaction with a UI [user interface] for making selections). Such a database can also be used to control inventory, marketing, manufacturing, send warnings or notices to sales staff, shipping and/or security, IT maintenance, promotions, etc. Further, the database can be a trigger to send such notification by, for example, e-mail or other forms of communication (i.e., electronic or non-electronic means).

As stated above, the Search Results window also contains a GUI (e.g., check box, 606) that can be activated to purchase selected items identified in the search (FIG. 11). The button 606, once activated, loads a shopping cart page which displays the item, quantity ordered, price and total for the amount of product ordered. Further, the page contains offers, services and advertisements that might be helpful to the user. The user may then cancel order (clear cart), recalculate order based on any discounts available, or proceed to checkout by activating the appropriate GUI (e.g., button).

Once the appropriate GUI is activated, a new web page is loaded and the user is directed to input user specific information for purchase and tracking in a customer field (dialog box).

### Example 7

### Representative Embodiments

Listed hereafter are representative embodiments of collections and methods.

1. A collection of matched biological reagents comprising at least 100 different biological reagents of each of at least two biomolecule classes and/or biological research product classes, wherein the isolated biological reagents of the collection are related to a target biomolecule or biomolecular pathway.

2. The collection of aspect 1, wherein the biological reagents are selected from the group consisting of antibodies, RNAi, nucleic acids, enzymes, proteins, cell culture media, detection products, separation media, microarrays, and the like.

3. The collection of any one of the preceding aspects, wherein the collection comprises at least 500 different isolated biological reagents of each of at least two biomolecule classes and/or biological research product classes.

4. The collection of any one of the preceding aspects, wherein the collection comprises at least 100 different isolated biological reagents of each of at least three biological research product classes.

5. The collection of any one of the preceding aspects, wherein the collection comprises at least 100 different isolated mammalian biomolecules.

6. The collection of any one of the preceding aspects, wherein the collection comprises at least 100 different isolated nucleic acids, at least 100 different isolated proteins encoded by the at least 100 different isolated nucleic acids, at least 100 different antibodies against the at least 100 different proteins, and at least 100 different recombinant cell lines comprising each of the at least 100 different isolated nucleic acids.

7. A method for selling an isolated biological reagent, comprising: presenting to a customer an input function for identifying a target biological molecule or target biological pathway; and presenting to the customer a purchasing function comprising links to purchases of at least 100 different individual or different combinations of matched biological reagents of a collection of matched biological reagents comprising at least 100 different isolated biological reagents of each of at least two biomolecule classes and/or biological research product classes, wherein the isolated biological reagents of the collection are related to the target biomolecule or biomolecular pathway.

8. A method for selecting an isolated biological reagent from a collection, comprising: inputting a search parameter into an input function; identifying at least 100 different individual or different combinations of matched biological reagents from a collection of biological reagents comprising at least 100 different isolated biological reagents of each of at least two biomolecule classes and/or biological research product classes, wherein the isolated biological reagents of the collection are related to the search parameter.

9. The method of aspect 8, wherein the search parameter is selected from the group consisting of a target biological molecule, a target biological pathway, a target biological pathway member, a disease, a disease pathway, and a disease pathway member.

10. The method of any one of the preceding aspects, wherein the biological reagents are selected from the group consisting of antibodies, RNAi, nucleic acids, enzymes, proteins, cell culture media, detection products, separation media, microarrays, and the like.

11. The method of any one of the preceding aspects, wherein the collection comprises at least 500 different isolated biological reagents of each of at least two biomolecule classes and/or biological research product classes.

12. The method of any one of the preceding aspects, wherein the collection comprises at least 100 different isolated biological reagents of each of at least three biological research product classes.

13. The method of any one of the preceding aspects, wherein the collection comprises at least 100 different isolated mammalian biomolecules.

14. The method of any one of the preceding aspects, wherein the collection comprises at least 100 different isolated nucleic acids, at least 100 different isolated proteins encoded by the at least 100 different isolated nucleic acids, at least 100 different antibodies against the at least 100 different proteins, and at least 100 different recombinant cell lines comprising each of the at least 100 different isolated nucleic acids.

The entirety of each patent, patent application, publication and document referenced herein hereby is incorporated by reference. Citation of the above patents, patent applications, publications and documents is not an admission that any of-the foregoing is pertinent prior art, nor does it constitute any admission as to the contents or date of these publications or documents.

Singular forms “a”, “an”, and “the” include plural reference unless the context clearly dictates otherwise. Thus, for example, reference to “a subset” includes a plurality of such subsets, reference to “a nucleic acid” includes one or more nucleic acids and equivalents thereof known to those skilled in the art, and so forth. The term “or” is not meant to be exclusive to one or the terms it designates. For example, as it is used in a phrase of the structure “A or B” may denote A alone, B alone, or both A and B.

Unless defined otherwise, all technical and scientific terms used herein have the same meanings as commonly understood by one of ordinary skill in the art to which this invention belongs. Although any methods and systems similar or equivalent to those described herein can be used in the practice or testing of the present invention, the methods, devices, and materials are now described. All publications mentioned herein are incorporated herein by reference for the purpose of describing and disclosing the processes, systems and methodologies which are reported in the publications which might be used in connection with the invention. Nothing herein is to be construed as an admission that the invention is not entitled to antedate such disclosure by virtue of prior invention.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A collection of matched biological reagents, comprising at least 100 different isolated biological reagents of each of at least two biomolecule classes and/or biological research product classes, wherein:
the isolated biological reagents of the collection are related to a target biomolecule, biomolecular pathway or disease pathway; and
the collection comprises at least 100 different isolated nucleic acids, at least 100 different isolated proteins encoded by the at least 100 different isolated nucleic acids, at least 100 different antibodies against the at least 100 different proteins, and at least 100 different recombinant cell lines comprising each of the at least 100 different isolated nucleic acids.
